(19) United States
(12) Patent Application Publication
(10) Pub. No.: US 2024/0415628 A1
(43) Pub. Date: Dec. 19, 2024

(54) EMBOLIC PROTECTION DEVICE DESIGNED IN PARTICULAR FOR TORTUOUS BLOOD VESSELS, ESPECIALLY CEREBRAL VESSELS

(71) Applicant: MIVI Neuroscience, Inc., Eden Prairie, MN (US)

(72) Inventors: John Wainwright, Foothill Ranch, CA (US); Joseph Marrocco, Costa Mesa, CA (US); Charles M T Gordon, Maple Grove, MN (US); Charles L. Anderson, Saint Paul, MN (US); Julia Holmstrom, Prior Lake, MN (US); Christian G. Monroe, San Diego, CA (US); Jack B. Sattell, Cambridge, MA (US); Brendan Gonzales, San Diego, CA (US); Pankaj Gupta, Maple Grove, MN (US); Englong Tan, Mahtomedi, MN (US)

(21) Appl. No.: 18/209,335

(22) Filed: Jun. 13, 2023

(51) Int. Cl. A61F 2/01

(52) U.S. Cl. CPC A61F 2/011; A61F 2/0108; A61F 2210/0057; A61F 2250/0097; A61F 2250/0098; A61M 2025/0004; A61M 25/0013; A61M 25/0023

(57) ABSTRACT

An embolic protection device with a flexible fiber-based filter element is described associated with an integrated guide structure. The integrated guide structure comprises a corewire within a hypotube having an uncut proximal section and a distal section having laser cuts through the hypotube wall. A corewire extends through the hypotube with a low friction channel, which can have a friction reducing coil between the corewire and at least a portion of the hypotube. A torque coupler restricts rotation of the corewire while allowing at least some sliding of the corewire within the hypotube that provides for actuating the filter element and for curving the laser cut hypotube. Torque coupler designs provide connection to the laser cut hypotube. The fiber bundle has an initial undeployed configuration with the fibers aligned and a deployed configuration with the fibers bent.

### FIELD OF THE INVENTION

The invention relates to fiber-based vascular filter designed to provide embolic protection, in which the device is supported on an integrated guiding device with a wire functioning as an actuation structure. The invention further relates to the design of the integrated guiding device to provide for pushability through tortuous vessel while providing access to narrow vessel, such as cerebral vessel.

### BACKGROUND OF THE INVENTION

A variety of procedures are performed with less invasive approaches to reach distant locations within a patient's body. Many of the procedures are performed within the cardiovascular system. For any of these procedures, a guidewire can be used to snake through the patient to position the tip of the guidewire at a desired location. A catheter and/or other medical devices can be positioned by sliding them over the guidewire to the appropriate location.

Many less invasive procedures create the possibility of emboli formation as a result of the procedure. Also, some procedures may be specifically initiated to capture and/or remove clots in a vessel. An embolus can be any particle comprising a foreign and/or native material, which enters the vascular system or other vessel of the body with potential to form a clot and cause occlusion of flow, e.g., blood flow. Emboli can be formed from aggregates of fibrin, blood cells or fragments thereof, collagen, cholesterol, plaque, fat, calcified plaque, bubbles, arterial tissue, and/or other miscellaneous fragments or combinations thereof. Loss of blood flow to surrounding tissue causes localized cell death or microinfarcts. Cerebral microinfarcts can cause stroke leading to confusion, disturbance of speech, paralysis, visual disturbances, balance disturbances and even death. In the heart, emboli can cause myocardial infarcts, i.e. heart attacks. Myocardial infarction refers to the death of a section of myocardium or middle layer of the heart muscle. Myocardial infarction can result from at least partial blockage of the coronary artery or its branches. Blockage of capillaries associated with the coronary arteries can result in corresponding microinfarctions/microinfarcs. Resulting impairments are frequently short term but can be permanent.

Ischemic strokes can be caused by clots within a cerebral artery. The clots block blood flow, and the blocked blood flow can deprive brain tissue of its blood supply. The clots can be thrombus that forms locally or an embolus that migrated from another location to the place of vessel obstruction. To reduce the effects of the cut off in blood supply to the tissue, time is an important factor. In particular, it is desirable to restore blood flow in as short of a period of time as possible. The cerebral artery system is a highly branched system of blood vessels connected to the interior carotid arteries. The cerebral arteries are also very circuitous. Medical treatment devices should be able to navigate along the circuitous route posed by the cerebral arteries for placement into the cerebral arteries.

### SUMMARY OF THE INVENTION

In a first aspect, the invention relates to an embolic protection device with a flexible fiber-based filter element comprising a corewire, a hypotube, a friction reducing coil, a torque coupler, a fiber bundle, and a distal tip. The corewire generally comprises a proximal section with a first diameter, a distal section having a second diameter less than the first diameter, and a tip section having a non-circular cross section over at least a portion of its length. The hypotube can comprise a proximal section free of laser cuts and a distal section having laser cuts through the wall of the hypotube. The corewire may extend through the hypotube with the proximal end and the distal end of the corewire extending from respective ends of the hypotube.

The friction reducing coil may be positioned between the corewire and at a portion of the hypotube distal section. The torque coupler may restrict rotation of the corewire and the hypotube at the torque coupler while allowing at least some sliding of the corewire within the hypotube.

The fiber bundle generally comprises a bundle of fibers, each fiber having a first end and a second end, a first attachment element, and a second attachment element. The first attachment element secures the first end of the polymer fibers and the second attachment element secures the second end of the fibers. The first attachment element is secured to the distal section of the hypotube. The fiber bundle has an initial undeployed configuration with the fibers aligned and a deployed configuration with the fibers bent. The corewire is in a proximal position relative to the hypotube in the undeployed configuration. The distal tip is secured with the second attachment element and/or to the corewire.

In a further aspect, the invention relates to an embolic protection device with a flexible fiber-based filter element comprising a corewire, a hypotube, a stake, a fiber bundle, and a distal tip. The corewire generally comprises a proximal section with a first diameter, a distal section having a second diameter, and a tip section having at least a segment having a non-circular cross section. The hypotube generally comprises a proximal section free of laser cuts and a distal section having laser cuts through the wall of the hypotube. The corewire extends through the hypotube with the proximal end and the distal end of the corewire extending from respective ends of the hypotube.

The stake generally comprises a central lumen with a non-circular cross section along the inner diameter. The non-circular cross section of the inner diameter is shaped to engage the non-circular cross section of the tip section of the corewire. The stake is interfaced with the corewire to prevent relative rotation of the stake and the corewire without limiting sliding of the corewire relative to the stake. The stake is secured with the hypotube to resist relative rotation of the stake and the hypotube.

The fiber bundle comprises a bundle of fibers each having a first end and a second end, a first attachment element, and a second attachment element. The first attachment element secures the first end of the fibers, and the second attachment element secures the second end of the fibers. The first attachment element is secured to the distal section of the hypotube. The fiber bundle has an initial undeployed configuration with the fibers aligned and a deployed configuration with the fibers bent and the corewire in a proximal position relative to the hypotube in the undeployed configuration. The distal tip is secured with the second attachment element and/or to the corewire.

In a further aspect, the invention relates to an embolic protection device with a flexible fiber-based filter element comprising a corewire, a hypotube, a fiber bundle, and a distal tip. The corewire generally comprises a distal end and a proximal end. The hypotube generally comprises a metal shaft with a lumen. The corewire extends through the lumen of the hypotube with the proximal end and the distal end of the corewire extending from respective ends of the hypotube.

The fiber bundle generally comprises a bundle of fibers each having a first end and a second end, a first attachment element, and a second attachment element. The first attachment element secures the first end of the fibers, and the second attachment element secures the second end of the fibers. The first attachment element is secured to the hypotube such that relative movement of the corewire and the hypotube can transition the filter cartridge from a low profile delivery confirmation to a deployer filtering conformation. The fiber bundle has an initial undeployed configuration with the fibers aligned and a deployed configuration with the fibers bent. The corewire is in a proximal position relative to the hypotube in the undeployed configuration. The distal tip comprises a distal coil secured with the second attachment element and/or to the corewire. The first attachment element generally comprises a first marker band having laser cut walls over at least a portion of its length. The first marker band is formed from a highly radiopaque metal.

In a further aspect, the invention relates to an embolic protection device with a flexible fiber-based filter element comprising corewire, a hypotube, a friction reducing structure, a torque coupler, a fiber bundle, and a distal tip. The corewire generally comprises a proximal section with a first diameter, a distal section having a second diameter less than the first diameter, and a tip section having a non-circular cross section over at least a portion of its length. The hypotube generally comprises a proximal section free of laser cuts and a distal section having laser cuts through the wall of the hypotube. The corewire may extend through the hypotube with the proximal end and the distal end of the corewire extending from respective ends of the hypotube.

The friction reducing structure generally comprises a polymer associated with the hypotube or a mechanical element having a low friction channel. The friction reducing structure may be located between the corewire and a portion of the hypotube distal section. The torque coupler may restrict rotation of the corewire and the hypotube at the torque coupler while allowing at least some sliding of the corewire within the hypotube.

The fiber bundle generally comprises a bundle of fibers each having a first end and a second end, a first attachment element, and a second attachment element. The first attachment element secures the first end of the polymer fibers and the second attachment element secures the second end of the fibers. The first attachment element is secured to the distal section of the hypotube. The fiber bundle has an undeployed configuration with the fibers aligned and a deployed configuration with the fibers bent and the corewire in a distal position in the hypotube in the undeployed configuration relative to the position in the deployed configuration. The distal tip is secured with the second attachment element and/or to the corewire. Advancing the corewire within the hypotube in a distal direction with the fiber bindle in an undeployed configuration results in curving of the hypotube along the distal section.

In a further aspect, the invention relates to a method for delivering an embolic protection device comprising a corewire, a hypotube, a friction reducing structure, a torque coupler, a fiber bundle, and a distal tip. The corewire generally comprises a proximal section with a first diameter, a distal section having a second diameter less than the first diameter, and a tip section having a non-circular cross section over at least a portion of its length. The hypotube generally comprises a proximal section free of laser cuts and a distal section having laser cuts through the wall of the hypotube. The corewire may extend through the hypotube with the proximal end and the distal end of the corewire extending from respective ends of the hypotube. The friction reducing structure generally comprises a polymer associated with the hypotube or a mechanical element having a low friction channel. The friction reducing structure is located between the corewire and a portion of the hypotube distal section. The torque coupler may restrict rotation of the corewire and the hypotube at the torque coupler while allowing at least some sliding of the corewire within the hypotube.

The fiber bundle generally comprises a bundle of fibers each having a first end and a second end, a first attachment element, and a second attachment element. The first attachment element secures the first end of the polymer fibers and the second attachment element secures the second end of the fibers. The first attachment element is secured to the distal section of the hypotube. The fiber bundle has an undeployed configuration with the fibers aligned and a deployed configuration with the fibers bent and the corewire in a distal position in the hypotube in the undeployed configuration relative to the position in the deployed configuration. The distal tip is secured with the second attachment element and/or to the corewire.

The method generally comprises advancing the filter cartridge through a patient's vasculature; and steering direction of advancement of the filter by pushing the corewire in a distal to proximal direction to curve the hypotube along a laser cut section and orienting the curve in a desired direction.

In another aspect, the invention pertains to a method for assembling an integrated guide structure comprising assembling components of the integrated guide structure and reflowing a thermoplastic elastomer jacket. The components generally comprise a hypotube, a corewire, and a central element. The hypotube may have laser cuts along at least a portion of the length of the hypotube that extend through the wall of the hypotube. The corewire may extend through a central lumen of the hypotube. The central element generally comprises a central hole through which at least a portion of the corewires extends through and an outer surface that fits within the central lumen of the hypotube. The thermoplastic elastomer jacket is reflowed over the exterior of the laser cut hypotube such that the thermoplastic elastomer extends through holes through the hypotube to engage the exterior of the central element to resist movement of the central element without restricting at least some movement of the corewire within the lumen of the hypotube.

### BRIEF DESCRIPTION OF THE DRAWINGS

FIG. 1A is a side view of a particular embodiment of an integrated embolism protection device and actuator.

FIG. 1B is a side view of the device of FIG. 1A with an actuator removed depicting a corewire extending beyond a proximal end of a hypotube.

FIG. 2A is a side view of the device of FIG. 1A following expansion of the embolism protection device.

FIG. 2B is a side view of the device of FIG. 2A with the actuator removed depicting relative motion of the corewire as it extends further beyond a proximal end of a hypotube.

FIG. 3 is a side view of an embodiment of a laser cut hypotube.

FIG. 4A is a fragmentary, enlarged view of an embodiment of a spiral laser cut hypotube and a sectional view of the same.

FIGS. 4B and 4C are photographic views of embodiments of the spiral laser cut hypotube of FIG. 4A depicting variations in pattern characteristics.

FIG. 4D is a fragmentary view of the distal end of a laser cut hypotube having interlocking cut segments.

FIG. 5A is a side view of an embodiment of a corewire.

FIG. 5B is a top view of the corewire of FIG. 5A.

FIG. 6A is a fragmentary, expanded sectional side view of an embodiment of a torque coupler.

FIG. 6B is a sectional view of the torque coupler of FIG. 6A taken along line B-B.

FIG. 6C is a sectional view of the torque coupler of FIG. 6A taken along line C-C.

FIG. 7A is a front view of an embodiment of a stake.

FIG. 7B is a side view of the stake of FIG. 7A.

FIG. 7C is a top view of the stake of FIG. 7A.

FIG. 8 is a side view of the stake of FIG. 7A on a corewire.

FIG. 9A is a fragmentary, expanded sectional side view of an embodiment of an integrated embolism protection device including a stake on a corewire within a laser cut hypotube with a polymer cover.

FIG. 9B is a fragmentary, expanded sectional top view of the device of claim 9A.

FIG. 9C is a sectional view of the device of FIG. 9A taken along the C-C line.

FIG. 10A is a side view of an embodiment of an inner coil.

FIG. 10B is a front view of the inner coil of FIG. 10A.

FIG. 11 is a fragmentary, expanded sectional side view of an embodiment of an integrated embolism protection device including an inner coil.

FIG. 12A is a sectional view of the device of FIG. 11 taken along the A-A line showing polymer flowing fully through laser cut slits in the hypotube.

FIG. 12B is a sectional view of the device of FIG. 11 showing polymer partially flowing into slits in the laser cut hypotube.

FIG. 13A is a side view of an embodiment of a laser cut marker band.

FIG. 13B is a front view of the laser cut marker band of FIG. 13A.

FIG. 14A is a perspective side view of a specific embodiment of a distal marker band with a fenestrated proximal end.

FIG. 14B is a side view of the distal marker band of FIG. 14A.

FIG. 14C is an end view of the distal marker band of FIG. 14A.

FIG. 14D is a side view of a specific embodiment of a proximal marker band with a step.

FIG. 14E is an end view of the proximal marker band of FIG. 14D.

FIG. 15A is a fragmentary, side view of a fiber cartridge in a delivery configuration mounted on a laser cut hypotube with enlarged sectional views depicting the mounting structure.

FIG. 15B is the fiber cartridge of FIG. 15A in a deployed configuration.

FIG. 16A is a perspective side view of a specific embodiment of a fiber cartridge that integrates a torque coupler component with a marker band.

FIG. 16B is a sectional side view of the fiber cartridge of FIG. 16A.

FIG. 16C is an end view of the proximal end of the device of FIG. 16B.

FIG. 16D is a fragmentary, side view of the fiber cartridge of FIG. 16A in a delivery configuration mounted on a laser cut hypotube.

FIG. 16E is the fiber cartridge of FIG. 16D in a partially deployed configuration with enlarged sectional views depicting the mounting structure.

FIG. 16F is a sectional view of the device of FIG. 16E taken along the F-F line.

FIG. 17A is a schematic side view of a deployed filter cartridge with four curved metal elements and with polymer fibers removed to show the metal elements.

FIG. 17B is a filter cartridge with three metal elements extended into pedal shapes and surrounded by bent polymer fibers.

FIG. 18A is a side view of an embodiment of a distal coil.

FIG. 18B is a front view of the distal coil of FIG. 18A.

FIG. 19A is a side view of a specific embodiment of a fiber cartridge.

FIG. 19B is a sectional view of the device of FIG. 19A taken along the B-B line.

FIG. 20A is a perspective view of an embodiment of a deep-grooved (surface capillary) fiber filament.

FIG. 20B is an end view of the deep grooved fiber filament of FIG. 20A.

FIG. 21A is a fragmentary, side view of a fiber cartridge with multiple sequential filter elements with a first filter element in a partially deployed configuration and a second fiber cartridge in a low-profile delivery configuration mounted to a laser cut hypotube.

FIG. 21B is a sectional view of the device of FIG. 21A taken along the A-A line.

FIG. 21C is a sectional view of the device of FIG. 21A taken along the B-B line.

FIG. 22A is a perspective view of an embodiment of an actuating tool for use with a corewire.

FIG. 22B is a sectional view of the device of FIG. 22A.

FIG. 23A a side view of an alternative embodiment of an actuating tool for use with a corewire.

FIG. 23B is a side view of the actuation tool of FIG. 23A with structure removed to expose internal features of the tool.

FIG. 24 is a fragmentary, side view of an embodiment of an integrated embolism protection device when the corewire is overextended.

FIG. 25A is a side view of a microcatheter.

FIG. 25B is a side view of an aspiration catheter system comprising a guide catheter with an aspiration catheter engaged with the guide catheter to form an aspiration lumen extending through the aspiration catheter and a portion of the guide catheter, as shown as transparent to allow visualization of structure within the guide catheter.

FIG. 26 is a view of a treatment system comprising an integrated embolism protection device, a stent retriever and an aspiration system with a guide catheter and a cooperating aspiration catheter.

FIG. 27 is a schematic drawing of a human patient with a treatment system as described herein entering the patient at the femoral artery and extending into a cerebral artery.

FIG. 28 is a fragmentary view of a section of vasculature with a guide catheter having its distal tip in a carotid artery and an integrated embolism protection device through a clot in a cerebral artery.

FIG. 29 is a fragmentary view of the cerebral artery of FIG. 28 with an integrated embolism protection device extending with its distal end past the clot and an aspiration catheter tip positioned in a proximal location.

FIG. 30 is a fragmentary view of the cerebral artery of FIG. 29 with a deployed fiber based filter.

FIG. 31 is a fragmentary view of the cerebral artery of FIG. 30 in which the clot or portion thereof has been pulled part of the way with the aspiration catheter using the filter to engage the clot.

FIG. 32 is a fragmentary view of the cerebral artery of FIG. 31 in which the clot or portion thereof have been pulled to the opening of the aspiration catheter.

FIG. 33 is a fragmentary view of the cerebral artery of FIG. 32 in which the aspiration catheter, clot or fragment thereof and the filter are being removed from the patient.

FIG. 34 is a fragmentary view of the cerebral artery of FIG. 30 in which the distal tip of the aspiration catheter has been advanced up to the clot.

FIG. 35 is a fragmentary view of the cerebral artery of FIG. 34 in which the clot or portion thereof have been pulled into the opening of the aspiration catheter.

FIG. 36 is a fragmentary view of the cerebral artery of FIG. 35 in which the aspiration catheter and the filter are being removed from the patient, in which the filter has been at least partially collapsed into a lower profile configuration and drawn at least partially into the lumen of the aspiration catheter.

FIG. 37 is a fragmentary view of an embolic protection device being delivered to a section of vasculature with a branched vessel.

FIG. 38 is a fragmentary view of an embolic protection device being delivered through an aspiration catheter to a section of vasculature with a branched vessel.

### DETAILED DESCRIPTION OF THE INVENTION

An embolic protection device has been developed to allow for improved access into narrow tortuous vessels, such as cerebral vessels. The embolic protection device comprises a corewire and a hypotube riding over the corewire with a fiber-based filter cartridge connected between them at or near their distal ends. The fiber based filter structure has the respective ends of the fibers attached such that movement of the corewire relative to the hypotube can transition the fibers from a low profile configuration parallel to the axis of the corewire to a deployed filter configuration with the fibers bent to extend radially outward from the corewire to form the filter structure. The design of the device is altered relative to early versions of similar devices allowing for greater and more consistent flexibility near the distal end while maintaining good core pushability. The changes in the hypotube, matched corewire, and attachment of the fiber cartridge provides for a strong structure that is pushable yet provides access to distant highly tortuous vessels. In particular, a significant portion of the hypotube can be laser cut to improve its flexibility. To maintain good surface properties and sliding of the corewire, a reflowed polymer jacket can be used while maintaining the flexibility, and a low friction channel along the interior of the laser cut hypotube, which can be formed with an inner coil, can be placed between the corewire and the laser cut hypotube to facilitate relative corewire movement. In view of the laser cut hypotube, a redesigned torque coupler can incorporate a stake anchored to the laser cut hypotube, or in other embodiments a component of the torque coupler is combined with a marker band or other component of a fiber attachment structure. In particular, a portion of the marker band may be shaped to form a component of a torque coupler. Also, changes to the attachment of the fiber cartridge can provide more consistent flexibility in the transition from the hypotube to the filter cartridge. The use of a laser cut radiopaque marker band can be used to provide some increase to the flexibility around the fiber attachment. A laser cut marker band can be applicable to other applications for flexible medical devices. Further embodiments of marker bands may also offer advantages to this and other devices. For example, fenestrations at an end of the marker band may improve the overall structure by allowing polymer, such as adhesive, to flow through the marker band to help bond the marker band to the corewire. In some embodiments, a stepped up portion at an end of a marker band may provide a desirable interface for joining with other components, such as a laser cut hypotube. The improved structure can be adapted for various procedures, such as thrombectomy procedures to remove clots, such as acute aschemic stroke events.

The use of suitable last cut hypotube, such as an intermittent spiral cut, provide for the possibility of manipulating the embolic protection device to alter the configuration of device that may be advantageous for delivery through the vasculature. In particular, if the corewire is pushed in a distal direction relative to the hypotube with the filter in an undeployed configuration, the attachment of the corewire to the hypotube prevents unconstrained movement of the corewire. The laser cut hypotube then bends in response to the force of pushing the restrained corewire in a distal direction. The bending of the laser cut hypotube allows for redirection of the tip of the device. The redirection of the tip can be used to steer the device during advancement in the vasculature and/or to mitigate ledge effects from structural features, such as branching, in the vessels. In some embodiments, the embolic protection device can be delivered in conjunction with an aspiration catheter for at least part of the delivery path of the device. Specifically, if the filter element is positioned at the distal opening of an aspiration catheter, the aspiration catheter can be less susceptible to ledge effects and the embolic protection device and the aspiration catheter are advanced together through the vasculature. Inducement of curvature of the laser cut hypotube of the embolic protection device can configure the filter at the opening of the aspiration catheter with an orientation that facilitates avoidance of a ledge effect.

To reduce the clinical effects of a clot within an artery, such as a cerebral artery, the clot can be removed, and it is correspondingly desirable to keep the time for removal short. Desirable clinical outcomes generally correlate with the length of time for treatment to reestablish flow block by the clot. For convenience, as used herein all arteries downstream from the interior carotid arteries are referred to as a cerebral artery, although the devices described herein can be used to access other arteries within a patient, such as peripheral arteries, coronary arteries and carotid arteries. Procedures involving the cerebral arteries can interface to varying degrees with the internal carotid arteries and are accessed through the external carotid arteries. The process of removal of the clot poses the challenge of tracking a device to the clot and engaging the clot to remove it. For at least a portion of the removal process, the clot can be drawn into a catheter or sheath to facilitate retention of the clot. While aspiration can be used alone or with mechanical engagement to remove the clot, it can be desirable to provide an embolic protection device to capture any released emboli resulting from attempts to clear a clot. Work to date suggests that this is a particular concern for harder clots that are more resistive to aspiration. Any portions of the clot that remains in the vessel or breaks off from the original clot can eventually flow downstream to block a smaller vessel with associated harm to the patient, especially since clots lodged in even smaller vessels may be more difficult or impossible to remove using a thrombectomy procedure. The placement of an embolectomy/thrombectomy device within a cerebral artery or similar tortuous vessel poses significant challenges due to the circuitous path through the vessels. Thus, a significant challenge is to safely place the device at a target location within the vessel, which is generally with the fiber filter element past the clot. As used herein, the term proximal is used in its conventional sense in this art to refer to the end of the structures toward the healthcare professional generally outside the vasculature, and the term distal has its usual meaning in this art of the end of the device extending furthest into the vasculature, in which relative terms track these meanings.

Fiber based filters have been found to result in very effective filtering within blood vessels. These devices can comprise a fiber mat formed of the fibers in a deployed configuration such that the fiber mat has the structure of a three dimensional filtration matrix. The three dimensional filtration matrix comprises effective pores with a distribution of sizes within the matrix. The pores with various sizes inside the matrix provide complex flow passages through the fiber mat to allow blood to pass through while effective retain emboli of various sizes. In some embodiments, the fibers are configured to be a non-woven bundle. Even after the deployment and formation of the fiber mat, the fibers remain unwoven. Filters formed from fiber bundles are described further in U.S. Pat. No. 7,879,062 to Galdonik et al., entitled “Fiber Based Embolism Protection Device,” incorporated herein by reference. The fiber bundles can comprise additional elements, such as thin metal wires, which can supplement the properties of the fiber bundle, see U.S. Pat. No. 10,463,386 to Ogle et al. (hereinafter the '386 patent), entitled “Thrombectomy Devices and Treatment of Acute Ischemic Stroke With Thrombus Engagement,” incorporated herein by reference. A commercial device with a fiber filtration matrix under these patents was sold by Medtronic under the tradename FiberNet®, and this device was described as having the smallest landing zone of commercial embolic protection devices, see Radvany. “Use of Embolic Protection Devices in Peripheral Interventions,” Interventional Cardiology Review 2017;12 (1): 31-5, incorporated herein by reference.

The integrated guiding device generally comprises a thin corewire and a tube, generally a hypotube, over the corewire. To provide improved flexibility while maintaining pushability, a significant distal section of the hypotube can be laser cut. The device can further comprise a torque coupling structure that couples torque on the tube with torque on the corewire, to provide for improved guiding of the integrated structure and to maintain the integrity of a fiber cartridge that transitions into the filter matrix. Torque couplers in integrated guide structures are described generally in U.S. Pat. No. 8,092,483 to Galdonik et al. (hereinafter the '483 patent), entitled “Steerable Device Having a Corewire Within a Tube and Combination with a Functional Medical Component,” incorporated herein by reference. The torque coupler may limit the relative motion of the corewire and overtube, although the filter cartridge similarly limits the relative movement. To provide for a hypotube with significant laser cutting at locations involving a torque coupler, a new torque coupler design is presented that is consistent with other design improvements. In the integrated structure, by parsing the overall diameter into a thinner corewire and an hypotube riding on the corewire, structure is introduced that can communicate between the proximal end and the distal end by longitudinally moving the corewire and the hypotube relative to each other. The hypotube rides over the corewire, and in the assembled device, the two elements generally cannot be fully separated without breaking the structure. Hypotube is a term used for hypodermic tube and in the art, is used to refer to small diameter medical grade tubing, usually but not necessarily formed from metal, such as stainless steel, Nitinol (nickel-titanium alloy) or the like. For the integrated guiding devices described herein, the corewire and hypotube (overtube) are generally made of metal. While the hypotube in the device design of primary interest comprises metal, in alternative embodiments, sections of the tube could comprise a polymer tube. To impart improved flexibility, especially near the distal end of the device, laser cut hypotube can be used to provide greater flexibility without unduly impeding pushability. To further assist with pushability, the integrated guide structure may transition from a first larger diameter toward the proximal section of the device relative to the distal portion, although desirable properties balancing puashability and flexibility are obtained with a section of solid hypotube and distal section of laser cut hypotube.

As explained in some detail below, the embolic protection device is generally used in conjunction with an aspiration catheter. Improved designs of aspiration catheters involve placement in a guide catheter to form an aspiration lumen extending in part through the guide catheter lumen. In this way, aspiration efficacy can be improved while having a aspiration catheter that can reach into smaller vessels beyond the reach of the guide catheter. Such a catheter is available from MIVI Neuroscience, Inc., the Q™ Catheter. The current embolic protection device is well suited for use in conjunction with the Q™ Catheter or comparable devices. These aspiration catheters provide excellent suction. The availability of an embolic protection device allows for more versatility with respect to application of aspiration while not inducing an undesirable risk from formation of emboli that can travel deeper within the vasculature. For example, intermittent or pulsatile aspiration may be more effective at removing harder clots but may have a small risk for emboli formation. Similarly, use of an abrasive device, such as a stent graft or the like, for harder clots may form emboli.

Alternatively or additionally, the embolic protection device can be used with other devices inserted into the vessel. For example, stent retrievers can be used with the protection provided by the embolic protection device. Similarly, a profusion catheter has been described, and prototypes are being tested, that can deliver flow in a reverse, i.e., proximal, direction to assist with dislodging a clot. See, U.S. Pat. No. 11,229,445 to Ogle, entitled “Hydraulic Displacement and Removal of Thrombus Clots, and Catheter for Performing Hydraulic Displacement,” incorporated herein by reference. While a hydraulic displacement catheter includes a balloon to restrict flow, subsequent deflation of the balloon can risk emboli release that can be captured by the embolic protection devices described herein. Some designs of the hydraulic displacement catheter provide for riding over a guidewire or similarly an integrated guiding structure.

While use of laser cut hypotube for all or a significant fraction of overtube provides significant advantages, this can also result in complexities. The laser cut hypotube generates surfaces that have more friction. Mitigating design changes have been found to compensate for the friction and to provide desirable performance of the device. In particular, a coil can be placed between the laser cut hypotube and the corewire. The coil is found to facilitate sliding of the corewire for actuation and de-actuation of the fiber filter. A thermoplastic elastomer, such as polyether block amide, can be reflowed over the outer surface to provide a lower friction exterior as well as to flow into the laser cut openings to stabilize the structure without a significant detrimental effect on the flexibility. The reflowed polymer jacket also resists stretching of the laser cut hypotube that can result in premature partial deployment of the fiber filter.

One objective in the embolic protection device design described herein is to allow for good pushability within the vessel to reduce difficulty for the health care professional to deliver the fiber bundle to the desired location within a patient's vessel. An uncut portion of the hypotube can provide greater column strength providing for better transfer of force along the length of the structure. While allowing for good pushability, the integrated guide structure should similarly provide a high degree of flexibility, especially at the distal end, which can be achieved with the laser cut hypotube. While particularly tight bends are found in the cerebral arteries, even getting into the carotid arteries from a femoral delivery point involves significant bends around the aortic arch to get into the left carotid artery or within the brachiocephalic artery to get into the right carotid artery. The femoral access is a standard and safe access point for catheter delivery, while other access points may also be suitable, such as from vessels in an arm. While these portions of the passage to the clot are through the guide catheter, the bends add to the collective sliding resistance both for the device itself and for the relative movement of the corewire.

When a significant portion of the hypotube is laser cut, it has been found that extending the corewire in a distal direction when the filter is not deployed results in a curvature of the integrated guide structure along the laser cut segment of the hypotube. The bending of the corewire can be accommodated by the laser cut hypotube based on appropriate stretching and contraction along the inner and outer curves. This bending may be useful to steer the device as well as for helping to avoid ledge effects.

For use, the fiber bundle generally is advanced past a clot. For placement of the fiber bundle past the clot, the fiber bundle advances past the various bends of the vessel before and possibly a short distance past the clot. The tip of the device extending past the fiber bundle should be pushable past the clot, which may take place through a microcatheter or the like. To provide the desired level of performance, the flexibility of the device around the fiber bundle can be significant. As described herein, the fiber bundle can be designed to provide improvements in the fiber bundle flexibility and, in particular, the consistency of the flexibility along the length of the fiber bundle to reduce kinking. For example, attachment points of the fibers in securing the fiber bundle can be made slightly shorter in extent and/or more flexible. Control of the device deployment is generally facilitated with radiopaque marker bands. In some embodiments, laser cutting of one or both marker bands on the respective sides of the fiber bundle can maintain desired functionality while improving flexibility. The anchoring portions for the fibers can also be shortened and an adhesive holding the fibers can be more elastic without compromising the fiber stability.

To provide its operation, the fiber bundle is attached at its distal end to the corewire and at its proximal end to the hypotube. The corewire and the hypotube can slide relative to each other longitudinally within constraints. The constraints can be provided by the attachments of the fiber bundle themselves, the actuator device, and/or one or more torque couplers that interface the hypotube and corewire along their length. The attachments should be secure to avoid disassembly of the fiber bundle within the patient. Also, it is desirable for radiopaque elements of the fiber bundle to provide visualization under x-ray imaging to provide information on the status of the fiber cartridge, in particular whether or not properly deployed. In some embodiments, upon full deployment of the filter cartridge, a distal marker band and proximal marker band are brought into contact, and this movement can be visualized by X-ray imaging in real time during the procedure.

It is desirable for at least one torque coupler to be relatively close to the proximal end of the filter cartridge. It has been known that an advantageous fiber filtration matrix is formed from a twisted unwoven fiber bundle. See the '483 patent cited above. The torque coupler facilitates forming the twist and maintaining the twist. A convenient design of the torque coupler can comprise flattening a section of the corewire and engaging the flat section of the corewire with a surface fixed to the hypotube that restricts rotation of the corewire relative to the hypotube without inhibiting sliding of the corewire within the lumen of the hypotube over an appropriate length. The length of the flattened section of the corewire can provide limits in one or both directions on the sliding of the corewire relative to the hypotube, but the limits may be more constrained by the fibers and their attachments themselves. Due to potential damage to the laser cut hypotube from altering its overall cylindrical shape, an approach of bending a hypotube or coil to engage a flat section of the corewire is not necessarily a desirable approach with the laser cut hypotube. An alternative feature fixed to the hypotube to resist rotation can be provided as an alternative to bending the laser cut hypotube if desired. In one embodiment, a stake is used that is slid over the corewire to rest between the corewire and hypotube, in which the stake has a keyway for passage of the corewire and a flattened or structured top surface to facilitate securing of the stake to the hypotube. The stake can be secured in place, for example, with reflowed polymer from the polymer jacket, although other comparable means may be suitable. In a further embodiment, a section of uncut hypotube can be interspersed between segments of laser cut hypotube to provide for an indentation without damaging a laser cut section, in which the uncut gap in the laser cut hypotube has an appropriate length along the tube, generally from about 2 mm to about 6 mm. Another approach for forming a distal torque coupler involves flattening a portion of a proximal marker band or other component associated with attachment of the proximal end of the fibers, in which the flattened part of the marker band/attachment component coincides with a flattened portion of the corewire. The marker band/attachment structure is secured to the hypotube, for example with laser welding, adhesive, or other suitable coupling approach, or combination thereof to inhibit relative rotation or other relative movement. If the hypotube is secured to the marker band to prevent movement, and an asymmetry provided to the marker band interfaces with a corresponding asymmetry of the corewire to form a torque coupler, the marker band can prevent any significant relative rotation of the corewire and hypotube while allowing for appropriate sliding movement of the corewire within the hypotube to engage or disengage the filter structure formed from the fibers in the fiber bundle.

The integrated guiding device can have a structure that can take advantage of these features with respect to manipulations at the fiber filter from the proximal end to transition the filter structure between configurations. At the same time, the outer surface of the hypotube can be used as a guide to introduce additional treatment devices that can be delivered over guidewires. As noted above, an aspiration catheter, in particular, can be delivered over the integrated guiding device.

In some embodiments, to reach desired locations in remote tortuous vessels, the integrated guiding device can be delivered through a microcatheter. The integrated guiding device can be sufficiently flexible to follow the microcatheter through branches of a patient's vascular system. The improved design of the integrated guiding device allows for the use of a smaller diameter microcatheter based on a narrower profile of the filter cartridge. If the embolic protection device is curved inside of a microcatheter, the microcatheter generally would be curved with the embolic protection device, and this can be advantageous for appropriate embodiments.

The integrated guide structures described herein introduce features that facilitate placement of the filter in a circuitous distant blood vessel, such as a cerebral artery. To provide access, the integrated guide structure should have appropriate pushability, while achieving requisite flexibility to wind through tortuous vessels, and stability to avoid kinking or misdeployment of the filter. To provide pushability, the proximal portion of the hypotube can have a structure without cuts through its wall. To provide desired flexibility, in some embodiments, the hypotube can have progressive laser cuts with increased flexibility toward the distal end.

Placement of the filter structure into a potentially curved narrow vessel can result in significant bending of the filter structure. The filter structure connects on its respective ends to the hypotube and the corewire, so the span of the filter structure between the fixed ends can be vulnerable to exposing the thin corewire to kinking forces. Providing a more flexible proximal connection of the filter cartridge to the hypotube can reduce the risk of kinking.

The fiber bundle comprises a plurality of fibers generally organized approximately uniformly around the circumference of the integrated guide structure. The unconstrained length of the fibers roughly correlates with the vessel size that can be effectively filtered since the deployed filter has bent fibers that extend out from the axis to the vessel wall, although the fibers should be somewhat longer to provide for the filter contacting the vessel wall to avoid any gaps in the filter matrix. The number of fibers in the bundle roughly correlates with the thickness of the three dimensional filtration matrix along the flow, but this also can depend on the vessel size. There can be a trade off with respect to ability to place the filter in the vessel if the filter cartridge has larger diameter and the desire to provide a desired filtration performance. In some embodiments, the length of the fibers can be roughly multiplied, such as doubled, with deployed fibers essentially bent twice, or otherwise multiplied to achieve a desired filtration matrix with fewer fibers and a corresponding thinner undeployed filer cartridge. Filter design can also comprise selection of the fibers and their corresponding properties. The fibers generally comprise polymer fibers that can provide good filtration performance while providing a less abrasive interface with the vessel wall, which may be relatively fragile. Surface capillary fibers have been found to provide desirable filtrations properties which seems associated with their high surface area. The fiber bundle can also comprise metal fibers, i.e., metal wires, for at least part of the fibers. To the extent that the deployed filter is pulled through the vessel, the metal fibers can provide mechanical stability to the filter matrix.

To allow for placement of the filter in the desired location of the vessel, a conventional guidewire can be first put in place, which can involve placing the tip of the guidewire past the occlusion. A microcatheter can then be placed over the guidewire, potentially with the distal end again extending past the occlusion, and then the guidewire can be removed. After the guidewire is removed, the integrated guide structure can be delivered through the microcatheter to place the filter element at the desired location in the vessel, generally with the filter a selected distance past the occlusion, and imaging can be used to confirm the location as well as the deployment of the filter. Then, the microcatheter can be removed. The filter can be transitioned to a deployed filtration configuration either before or after removal of the microcatheter from the vessel.

Other designs of embolic protection devices for cerebral vessels are described in U.S. Pat. No. 8,814,892 to Galdonik et al. (hereinafter the '892 patent, entitled “Embolectomy Devices and Methods for the Treatment of Acute Ischemic Stroke,” incorporated herein by reference. The '892 patent provides alternative strategies to overcome the flexibility concerns for delivery of the filter cartridge to the desired location in the cerebral arteries. The approaches herein provide convenient approaches to improve deliverability of the filter cartridge with convenient delivery procedures.

The embolic protection devices described herein provide for more routine manipulations for physicians undertaking the procedure to treat clot removal in remote circuitous vessels, especially for the treatment of acute ischemic stroke. In general, these procedures can involve an aspiration catheter at least for the retrieval of the filter, and additional medical devices can be involved, as described further below. In some embodiments, use of an aspiration catheter provides an important role for clot removal. The deployed filter can provide embolic protection through the selected procedures, and generally the filter is removed under aspiration at the end of the procedure. It can be desirable to pull the deployed filter toward the aspiration catheter to sweep any remaining thrombus or emboli to the aspiration catheter for removal. In some embodiments, the aspiration catheter is moved up to the filter matrix with the deployed filter stationary. At the end of the procedure, the filter can be transitioned to a collapsed configuration for removal into the aspiration catheter, or the deployed filter can be moved with the aspiration catheter in a deployed or partially extended configuration. In any method, the improved design of the catheter provides for more convenient manipulation of the filter structure in highly circuitous remote vessels, such as cerebral arteries.

### Embolic Protection Devices

Integrated guide structures with a distal filter are described in the following with the introduction of structural features that provide for enhanced delivery capability for placement of the filter into highly twisting blood vessel, especially remote blood vessels. A significant feature of the improved flexibility is the incorporation of laser cut hypotube for a distal section of the hypotube with uncut hypotube generally providing a proximal section of the hypotube. Design adaptations are introduced to provide desired functionality consistent with the laser cut segment of the hypotube. In particular, a coil can be introduced between at least a portion of the corewire and the laser cut hypotube to assist sliding of the corewire relative to the hypotube, and a new torque coupling structure is provided to interface with the laser cut hypotube. A thermoplastic elastomer can be reflowed to form a polymer jacket over the laser cut hypotube to provide a lower friction exterior surface while also extending into and possibly through the holes of the laser cut hypotube without significantly decreasing flexibility. In some embodiments, one or more laser cut radiopaque marker bands can improve flexibility of around the fiber cartridge. Additional changes to the attachment of the filter to the respective parts of the integrated guide structure can improve flexibility.

Slots extending into or through the wall of a catheter can be used generally to increase the flexibility of a catheter, other medical hypotubes or the like. In the present context, the overtube of the integrated guide structure is formed from a hypotube with laser cutting over a selected portion of the distal part of the device. In general, it may be desirable to have different flexibilities at different locations along a catheter or other medical tube. Specifically, it can be desirable to have increased flexibility toward the distal end of the catheter for steering of the device and other similar control aspects. To achieve these different flexibilities at different locations, the laser cutting can be correspondingly transitioned to provide the changes in flexibility.

The circuitous nature of blood vessels can introduce friction that inhibits directing the filter cartridge to a target location. Thus, the integrated guide structure is designed in the embodiments herein to be more pushable. But the features that provide a more pushable shaft are correspondingly less flexible, which make it difficult to navigate twisting vessels as well as difficult to enter small vessels. Thus, the distal end of the catheter is designed to be more flexible, while consistent with the pushable nature of the device based on the structural transitions along the length of the device. The circuitous nature of vessels not only makes it difficult to place the filter at the target location, but the vessel shape potentially can apply significant torque on the filter structure during movement through the vessel due to bending. The thin core wire can be susceptible to kinking due to bending at the filter landing zone, but the designed described herein provide appropriate level of support.

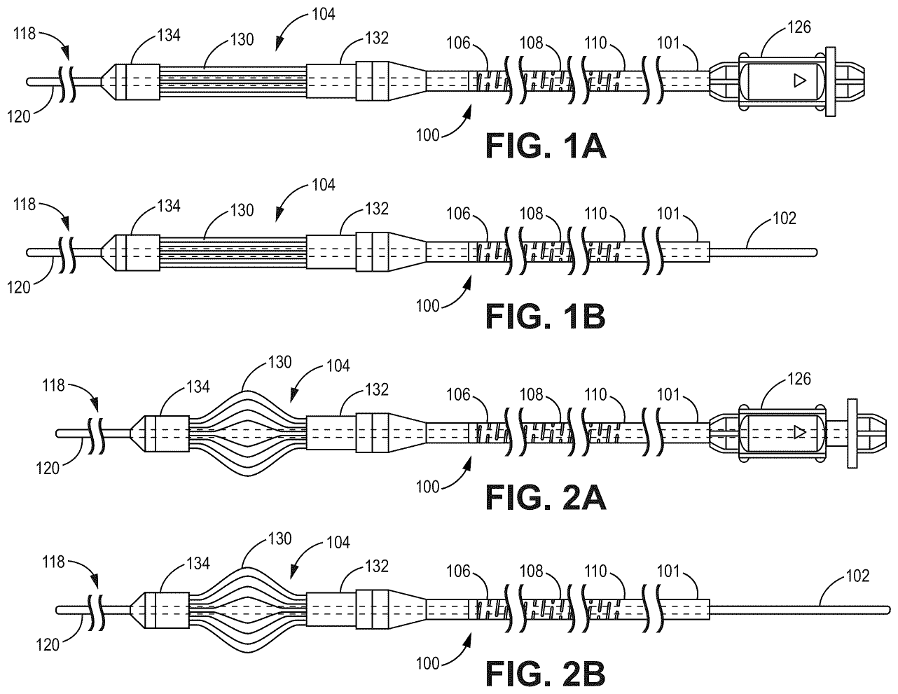

An embodiment of an integrated guide device 100 is shown in FIGS. 1 and 2. The integrated guiding device comprises a hypotube 101, a corewire 102, and a fiber cartridge 104. The integrated guide device can comprise an inner coil between a portion of the hypotube and the corewire and a polymer overcoat covering the hypotube or a portion thereof. The inner coil and polymer overcoat are discussed in further detail below. Referring to the side view in FIG. 1A, hypotube 101 has one or more laser cut sections, which are depicted in the figure as three sections 106, 108, 110, toward its distal end along with an uncut section 112 at the proximal end. Laser cut sections 106, 108, 110 may utilize different patterns in order to provide hypotube with different flexibility characteristics along different portions of the tube. Different numbers of laser cut sections and transition sections can be used, as described further below. Further details regarding ranges of laser cut patterns and hypotube flexibility are discussed below. Tip section 118 extends distally at the distal end of fiber cartridge 104. Tip generally comprises a coil connected with corewire 102 with solder or the like at distal end 120, although other blunt terminating structures can be used

Referring to FIGS. 1A and 1B, embolism protection device 104 is depicted in an undeployed configuration. Fiber bundle 104 comprises a bundle of fibers 130 attached at a first attachment 132 and a second attachment 134. FIG. 1B depicts the device of FIG. 1A with an embodiment of an actuator 126 removed, thereby exposing the proximal end of the corewire extending proximally beyond a proximal end of the hypotube. Various embodiments of actuator designs are described further below. Referring to FIGS. 2A and 2B, the device of FIG. 1 is depicted with the embolism protection device of FIG. 1 in a partially deployed configuration. FIG. 2A depicts a portion of actuator 126 extending in the proximal direction signifying the relative movement of the corewire relative to the hypotube. As seen in FIG. 2B, with the grip removed, when the embolism protection device is deployed, the portion of the corewire extending beyond the proximal end of the hypotube is lengthened.

Hypotube 100, corewire 102, and tip 118 can independently be formed from stainless steel, titanium, nitinol or other medical grade metals, although other suitable materials can be used such as a super elastic and/or shape memory material such as NiTi, NiTiCo, NiTiCr, Ti Beta or higher radiopacity superelastic material, such as MoRe alloy or W26Re alloy.

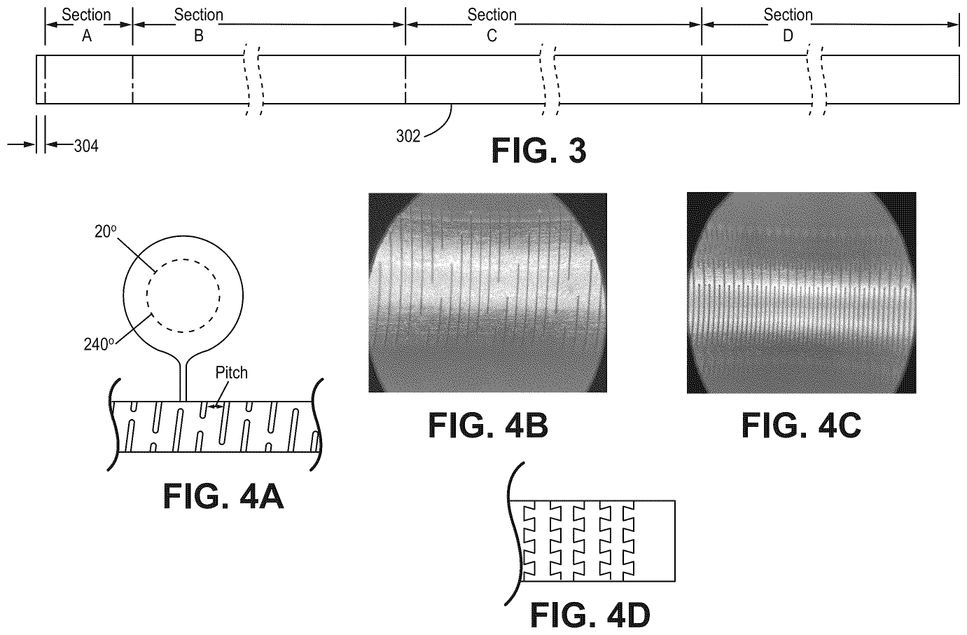

Referring again to FIG. 1, a selected degree of flexibility is introduced by a plurality of slots at laser cut sections 106, 108, 110. In some embodiments, the slots have at least a portion of their orientation along a transverse direction. Slots are a cut through the material of hypotube 100. The catheter/tube generally has an axis that orients the longitudinal direction of the catheter in a linear configuration. The transverse direction is perpendicular to the longitudinal axis such that slots with a transverse component of their orientation are not aligned only along an axial direction. In particular, an intermittent spiral cut can be effectively used and conveniently formed with existing laser cutting technology, in which a spiral cut is primarily circumferentially oriented with a small transverse component. In general, a slot can have any shape and not just a narrow elongated shape, although a thin elongated shape can be convenient. In general, the selection of laser cut sections can be made to achieve desired amounts of flexibility. Referring to FIG. 3, an embodiment of a laser cut hypotube 302 is divided into four distinct sections: Section A, Section B, Section C, and Section D, and an uncut distal segment 304. Sections A, B, and C can also have transitional cut patterns sections to transition between two adjacent sections with a consistent laser cut pattern. Transitional sections may have a generally linear progression across the section from a first pattern to a second pattern. They could also be stepwise or have non-repeating sections, as desired. The overall transition of the laser cuts are intended to involve desired transition of flexibility of the catheter at the distal end relative to the flexibility at the proximal end.

A schematic view of a spiral laser cut hypotube is shown in FIG. 4A with pitch noted along with enlarged photographic views in FIG. 4B (1.5 cuts per revolution (CPR)) and 4C (1.85 CPR). To achieve the desired flexibility of the hypotube, the pitch can range from about 0.0015 in to about 0.025 in, and different sections can have adjusted pitch to vary the flexibility to balance pushability and flexibility. The pitch is the edge-to-edge distance between adjacent cuts. A smaller pitch generally is associated with greater flexibility. The cuts per revolution (CPR) can generally range from about 1.25 to about 5. The angle of a cut can correspondingly range from about 20° to about 200°, and the range of uncut angle can be from about 10° to about 120°. To transition from a more flexible distal end to a less flexible proximal end, the pitch and/or the CPR (with corresponding decrease in cut angle and/or increase in uncut angle) can increase to decrease flexibility. In some embodiments, the catheter can have two or more distinct cut regions, such as 3, 4 or more than 4, which may include fixed cut regions optionally connected by a transition region where the parameters gradually change from one set of parameters to another. A person of ordinary skill in the art will recognize that additional ranges of cutting parameters within the explicit ranges above are contemplated and are within the present disclosure.

Cut regions may have specific values of cut and uncut angles that are fixed or vary across the region. In some embodiments, continuous cuts may be used with an interlocking pattern such that the cuts prevent elongation under tension. Interlocking segments can comprise more complex patterns in which the interlocking relationship discourages any lateral displacement along the axis since the edges of adjacent patterns block such movement without more extreme distortion of the pattern. An example of an interlocking pattern is shown in FIG. 4D. In some embodiments, multiple transition sections can be adjacent each other with different transition parameters with or without necessarily any fixed cut sections. In alternative or additional embodiments, essentially abrupt changes in LCH parameters can take place at adjacent fixed parameter cut regions. The cut portion of hypotube can be subjected to a manual stretching process, which can further increase the flexibility of the section of hypotube. For example, a distal end section can be manually stretched to further increase flexibility. In some embodiments, multiple hypotube diameters could be used to create a stepped assembled hypotube. The individual hypotubes could be laser cut prior to or post welding, soldering, sintering together or other joining methods a person of ordinary skill in the art will recognize. The properties of the laser cuts, the thickness of the hypotube, the hypotube metal (e.g., stainless steel, titanium, nitinol), the polymer jacket properties, other components contribute the resulting flexibility of the device.

The slight stretchability of the laser cut hypotube with appropriate laser cuts can be exploited to provide for curvature of the structure. As noted above, after assembly, if the hypotube is advanced in a distal direction relative to the hypotube with the filter in an undeployed configuration (the opposite movement to deploy the filter), the laser cut hypotube bends. The degree of bending can be significant, such as 180 degrees or more. This bending of the hypotube can exploited in various situations for delivery of the device in the vasculature. The center of curvature for the bend generally depends on the specifics of the device design, but generally the bending can occur toward the center of the laser cut section of the hypotube along the length of the device.

While most or essentially all of the hypotube can be laser cut, generally pushability is improved if the proximal portion is uncut. Generally, in the laser cut segment of the hypotube, at least about 90% of the length is laser cut, which refers to some removed metal around a circumference taken perpendicular to the axis. For devices designed for delivery from a femoral artery, the distal cut section can range from about 10 percent to about 50 percent, in further embodiments from about 15 percent to about 47 percent, and in other embodiments from about 18 percent to about 45 percent of the length of the hypotube with the remaining portions of the hypotube being a proximal uncut section. For delivery of the device through a different vessel, such as an artery in the arm, the appropriate portion of laser cut hypotube can be different, generally less. The cut section of the laser cut hypotube can be further characterized by its length independent of the entire lengths of the hypotube. The distal (laser cut) portion of the hypotube can be at least about 5 inches, in further embodiments from about 8 inches to about 30 inches, and in other embodiments from about 10 inches to about 25 inches. The uncut section can have incidental laser cuts without significantly altering the nature of the section, and the laser cut section can have various domains including potential, relatively short uncut sections and short uncut section at the end of the hypotube for stability, as noted above. A person of ordinary skill in the art will recognize that additional ranges of laser cut hypotube extent within the explicit ranges above are contemplated and are within the present disclosure.

The slots can be cut into the catheter/tube using any practical approach, and as used herein, reference to a laser cut does not limit the method for forming the cuts but refers to the nature of the cuts as being suitable for laser formation. Suitable cutting techniques include, for example, mechanical cutting, electrostatic discharge machining (EDM), cutting with high pressure fluids, chemical etching and laser cutting. Laser cutting can be particularly efficient for the formation of a significant number of precision cuts using automated control, especially cuts that penetrate through the catheter/tube to the inner lumen. Laser cut hypotubes according to appropriate parameters are available from Resonetics, LLC.

In a prototype embodiment, hypotube 302 has an inner diameter of about 0.0160 inches and an outer diameter of about 0.0200 inches and a spiral cut over specified sections. In the prototype, section A has a length of about 0.1 inches. The cut pattern for section A has a constant pitch of 0.0035 inches, cuts transitioning from 114 degrees at distal end to 129 degrees at a proximal end, uncut portions transitioning from 30 degrees at a distal end to 15 degrees at a proximal end, and a consistent 2.5 cuts per revolution (CPR). Section A includes an uncut distal tip 304 extending 0.0035 in. Section B has an axial length of about 5.57 inches. The cut pattern for section B has a pitch transitioning from 0.0035 inches at a distal end to 0.005 inches at a proximal end, cuts transitioning from 129 degrees at a distal end to 115 degrees at a proximal end, uncut portions transitioning from 15 degrees at a distal end to 29 degrees at a proximal end, and a consistent 2.5 CPR. Section C has an axial length of about 12.99 inches. The cut pattern for section C has a pitch transitioning from .0035 inches at a distal end to 0.011 inches at a proximal end. The cut pattern for section C has transitions from 83 degrees at a distal end to 35 degrees at a proximal end, uncut portions transition from about 19.86 degrees at a distal end to about 67.86 degrees at a proximal end, and consistent 3.5 CPR across the entire section. Section D is uncut and has an axial length of about 52.84 inches.

It may be desirable to coat or line a laser etched hypotube to provide appropriate surfaces for friction reduction or other desirable purpose. A low friction channel along the interior of the laser cut hypotube can provide for continued ability to slide the corewire within the hypotube. A liner may be placed on the outside of the hypotube, the inside of a hypotube, or both. For the formation of an integrated guiding device, it can be desirable to form a jacket of a thermoplastic elastomer that is reflowed after mounting over the exterior of the laser cut hypotube. Suitable polymers include, for example, polyether-block-amide, such as PEBAX®, thermoplastic polyurethanes, certain styrene block copolymers, mixtures thereof and the like Some specific polymers include, for example, such as NeuSoft thermoplastic polyurethane from Avient, Pellethane® thermoplastic polyurethanes from Lubrizol, or Tecoflex™ thermoplastic polyurethanes from Lubrizol. Suitable polymers may have a range of Shore Durometer values from about 25A to 75D (at least 25 A and no more than 75D) and in further embodiments from about 35A to about 40D, and in some embodiments from about 35A to about 85A. The polymer jacket can be varied along the length of the hypotube to have a softness value of the polymer jacket, expressed as a Shore Durometer Value, corresponding to a softer value toward the distal end of the hypotube. Shore durometer values are generally provided by material suppliers, and Shore Durometer values are obtained with appropriate equipment based on ASTM protocols. The A standard is generally used for softer materials than the D standard, but there is overlap and approximate relationships can roughly associate values for many materials in the different scales, which each extend from 0 to 100. With an appropriately selected polymer, the polymer flowing into or through the laser cut openings may not significantly alter the device flexibility. In some embodiments, the penetrating polymer can be used to maintain a stake providing torque coupling, as described below. At the location of the inner coil, polymer flowing through the laser cut opening can engage the coil to avoid penetrating to the corewire. Through appropriate placement of the component, reflowed polymer does not engage the corewire and does not restrict sliding movement of the corewire. The thermoplastic polymer jacket may or may not extend over the uncut proximal section of the hypotube, and the uncut portion of the hypotube may be covered with an alternative polymer jacket or coating. The uncut portion of the hypotube may also have an internal coating or liner to reduce friction. A liner or jacket integrated to the hypotube may not add significantly to the thickness of the hypotube permits the hypotube to retain the enhanced flexibility gained through the laser cuts.

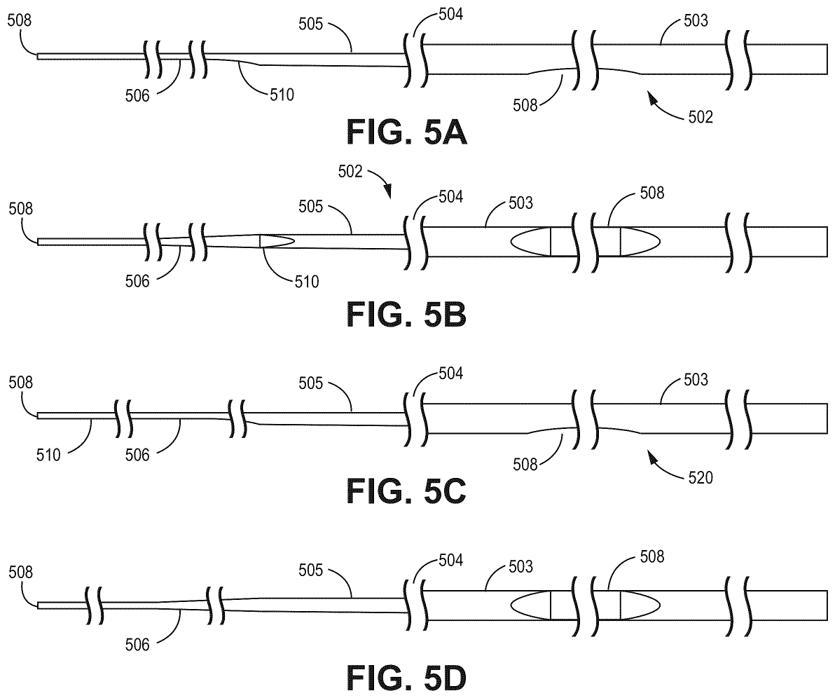

FIGS. 5A-5D show views of two different embodiments of corewires designed for use with a laser cut hypotube. These corewire designs are intended for use with a “stake” to provide torque coupling. As used in this context, a “stake” refers to a distinct element secured to the hypotube to provide a torque coupling function without inhibiting sliding of the corewire to deploy the filter. The corewire in FIGS. 5A and 5B is intended for use with a “stake” based torque coupler that is placed internal to the hypotube lumen, and the corewire of FIGS. 5C and 5D are intended for use with a “stake” based torque coupler integrated into a marker band on the proximal end of the filter cartridge such that the marker band becomes a stake for the torque coupler. Referring to FIGS. 5A and 5B, corewire 502 is a metal wire comprising portions of varying diameters and/or cross-sectional shapes. In the assembled device, corewire 502 extends essentially from a distal coil through the length of the hypotube and extends out from the proximal end of the hypotube. Corewire 502 has several distinct sections for assembly with the various components. A proximal section 503 has a larger diameter, and this section extends past the proximal end of the hypotube and through at least most of the length of the uncut hypotube. If the uncut segment of hypotube has sections with different diameters, proximal section 503 of corewire 502 can correspondingly have additional sections with corresponding diameter changes.

As shown in FIGS. 5A and 5B, corewire 502 comprises an intermediate section 505, and a transition section provides for transitioning between intermediate section 505 and proximal section 503 with a relatively sharp change or gradual taper, as appropriate. Intermediate section 505 has a smaller diameter to accommodate an inner coil between the hypotube and the corewire. A second tapered section 506 tapers towards distal tip 508 for the attachment of a fiber bundle for the filter. A thinner corewire at filter cartridge allows for a lesser profile for a particular number of fibers, while the diameter of distal tip 508 should be large enough to provide desired strength and kink resistance. As described below, a distal coil is mounted at the end of the corewire 502.

Generally, corewire 502 comprises one or more flattened or otherwise shaped key portion to provide for torque coupling with the hypotube. As shown in FIGS. 5A and 5B, corewire 502 has modifications for an optional proximal torque coupler and a distal torque coupler. Flattened section 508 forms a portion of an optional torque coupler discussed in further detail below. Flattened section 508 is a ground section that removes the cylindrical symmetry of the corewire around the axis with a flat or otherwise altered shape that provide for engagement to limit or eliminate relative rotation of the corewire and hypotube at the torque coupler. Flattened section 508 engages a mated indentation in the hypotube for torque coupling as described below. For construction of a distal torque coupler as described below, intermediate section 505 comprises a shaped key portion 510, such as a flattened portion, that is designed to engage a stake placed over the corewire. Shaped key portion 510 extends to the distal end of corewire 502 or is further sufficiently tapered at second taper section 506 and distal tip 508 to render the key shape irrelevant since the stake should be able to slide over the corewire from its distal end to near the proximal end of shaped key portion 510. Corewire 502 generally is covered with a polymer coating, such a polytetrefluoroethylene or a silicone polymer, to reduce friction for sliding motion of the corewire within the hypotube.

The embodiment of corewire 520 in FIGS. 5C and 5D is intended to engage a distal torque coupler that interfaces with the proximal end of the fiber cartridge. This is described below as involving a section of the marker band. The basic structure of corewire 520 in FIGS. 5C and 5D is essentially the same as corewire 502 in FIGS. 5A and 5B except that shaped key portion 510 can be shifted in a distal direction along the corewire. The completed design of the torque couplers are described below.

While the corewire and hypotube are intended to have at least limited, relative longitudinal movement to control fiber cartridge deployment, the longitudinal movement of the corewire within the hypotube is limited by several constraints. Attachment of the fiber cartridge on its respective ends to the hypotube and the corewire limit the range of relative motion of the corewire and hypotube without damaging the device. The structure of the one or more torque couplers may also provide constraints on the longitudinal movement of the corewire within the hypotube. Two alternative designs of a torque coupler that are particularly suitable for placement overlapping the laser cut hypotube are described in the following, and the corewire description above is consistent with on these torque coupler designs,

As noted above, the embolic protection device generally has one or more torque couplers. Torque couplers couple the rotational motion of the corewire with the hypotube. To maintain a desired twist in the fiber cartridge and effective deployment of the filtration matrix, it is desirable to have a torque coupler near the distal end of the device. A specific torque coupler design for placement at or adjacent regions of a laser cut hypotube are described below. Details of particular aspects of potential twists are also described further below. It may be desirable to include one or more additional torque couplers at more proximal positions of the device, which generally would be at positions with uncut hypotube, and may be based on previously developed torque coupler designs.

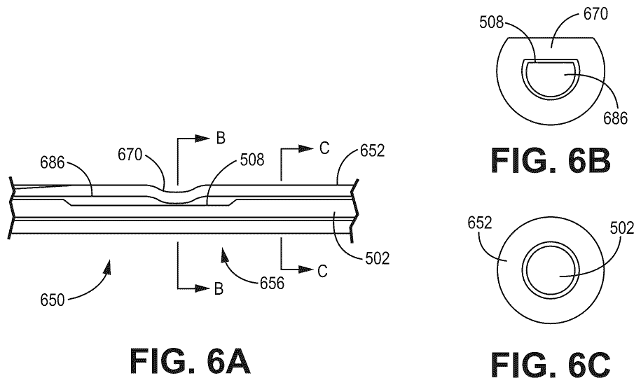

Torque couplers for use with an uncut hypotube can use designs originally found in the '483 patent cited above. Referring to a fragmentary view in FIG. 6, a specific embodiment of a torque coupler 650 is depicted that can be formed around flattened section 508 of corewire 502 shown in FIGS. 5A and 5B. Torque coupler 650 comprises corresponding structural features in hypotube 652 and corewire 502 that interface to form the torque coupler. Hypotube 652 comprises notch 670 that forms a portion of the torque coupler 650. Generally, the notch 670 is located in an uncut portion of a laser cut hypotube and represents a bending of the wall of the hypotube. Corewire 502 has a flattened section 508 to interface with notch 670. Notch 670 can slide along the extent of flattened section 508 while providing desired torque coupling.

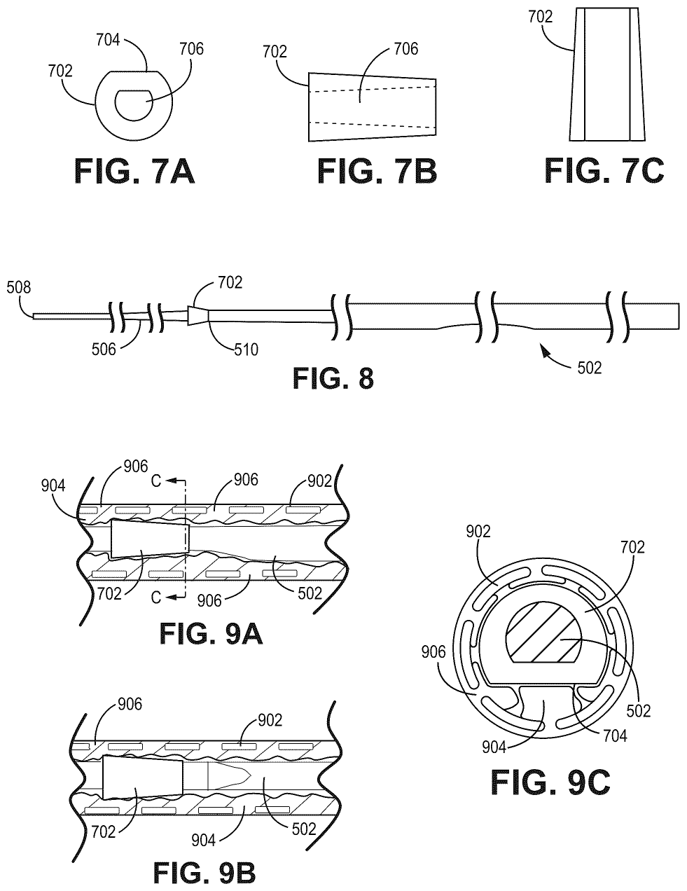

Referring to FIGS. 7-9, an embodiment of a distal torque coupler is shown with a specific design that functions desirably in a location with laser cut hypotube. A stake 702 may be used as a component of a torque coupler with a non-cylindrical hole engaged with an intermediate section of corewire 502 having a correspondingly shaped cross sectional shape to engage the non-cylindrical hole. Due to potential instability of the laser cut hypotube if it were indented, the stake is used to provide a stationary feature with a hole having an engagement surface to engage a corresponding segment of corewire 502 to resist relative rotation of corewire 502 and laser cut hypotube 302 without preventing sliding of corewire 502 relative to laser cut hypotube 302. Referring to FIGS. 7A-7C, stake 702 can have a generally circular exterior cross section over a portion of its circumference with an engagement surface 704 deviating from the circular shape to provide an engagement surface to enable stake 702 to be anchored in place, which is depicted in FIG. 7A as a flattened surface. Stake 702 also comprises a hole 706 with a non-circular cross sectional shape to provide an engagement surface for torque coupling and through which key shaped portion 510 of corewire 502 passes. While a flat surface is a convenient shape for engagement surface 704, any shape that allows for resistance to rotation can be suitable. The basic function of stake 702 is to lock relative orientation of corewire 502 and hypotube 302, so the stake is fixed to hypotube 652 such that locking relative orientation of stake 702 and corewire 502 correspondingly locks relative orientation of hypotube 302 and corewire 502. A hole with a non-circular cross-sectional shape through the stake engages a matched section of corewire 502 in a lock and key type fitting that does not prevent sliding of the corewire relative to the stake/hypotube. Other structures limit the extent of the range of corewire sliding through stake 702.

In general, it is desirable for stake 702 to have a small length along the corewire so that it does not significantly influence flexibility of the integrated guide structure. To provide suitable mechanical strength with its small size, stake 702 can be made from metal or similar mechanically strong material. While various techniques can be used to form stake 702, a convenient approach involves laser cutting a metal sheet to form stake 702 with a length corresponding to the metal sheet thickness. With existing laser cutting technology, such processing results in a particular, slightly tapered shape, which is not functionally needed, but correspondingly does not introduce any functional concerns. With a laser cut stake 702, the outside wall of stake 702 may have a slightly widening taper from the proximal to distal ends, while the opening 706 may have a slightly narrowing taper in the same direction. Accordingly, a thickness of the stake wall may slightly increase over the length of the stake, from the proximal to distal to end.

Referring to FIG. 8, stake 702 is depicted mounted on the corewire 502. The opening 706 of stake 702 may be slid over the distal end 508 of corewire 502. Stake 702 may be advanced over tapered section 506 of corewire 502 to shaped key portion 510. At shaped key portion 510, stake 702 has a fixed angular position. Hypotube 302 is positioned around stake 702 and reflow of the polymer jacket fixes stake in position.

Referring specifically to FIG. 9A, a fragmentary, expanded sectional side view depicts stake 702 mounted on corewire 502. Stake 702 and corewire 502 are positioned within a lumen in laser cut hypotube 902. Laser cut hypotube 902 has a polymer overcoat 904. Polymer overcoat 904 is shown extending through laser cut slots 906 in hypotube 902, which can be achieved, for example, by heating the polymer. Polymer overcoat 904 may surround stake 702, thereby securing it in position and preventing it from dislodging or sliding distally relative hypotube 902 over the tapered portion of the corewire. Referring to the cross-sectional view in FIG. 9C, polymer overcoat 904 fills in around the entire stake 702, and particularly densely around the engagement surface 704 of the stake 702, thereby forming a torque coupler. Rather than being able to rotate freely within laser cut hypotube 902, rotational force applied to a proximal portion of corewire 502 causes stake 702 to rotate, which, in turn, rotate the portion of laser cut hypotube 902 surrounding the embedded stake 702 such that there is no relative motion of hypotube 902 relative to corewire 502 at the stake.

As noted above, an alternative embodiment of a distal torque coupler for use for interacting with a laser cut hypotube can involve a marker band or other component of a fiber attachment structure. For these embodiments, a flattened section of the core wire can be included in a distal section of the corewire adjacent where fiber attachment is performed. Such an embodiment of the filter cartridge is discussed in detail below.

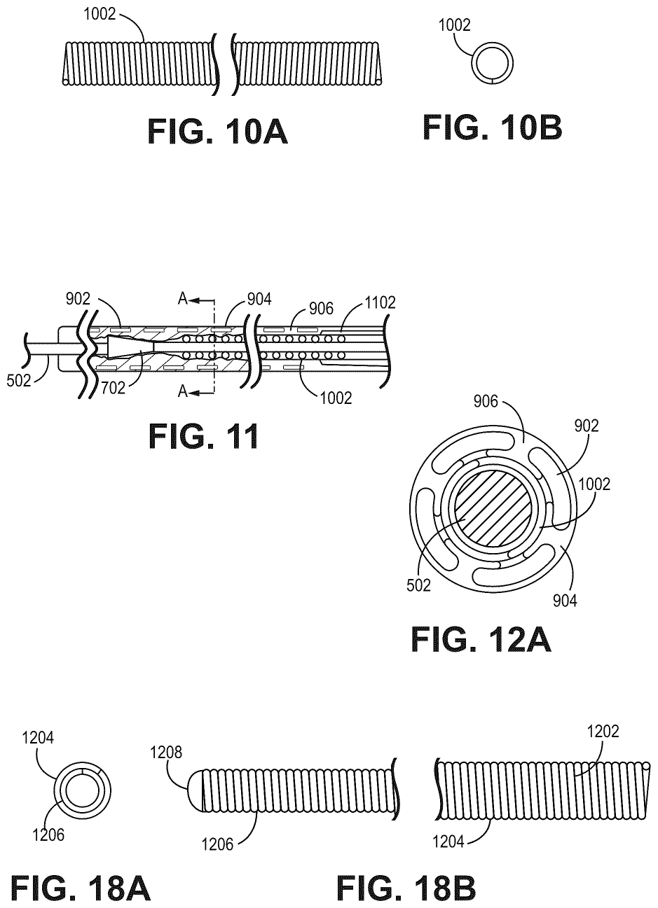

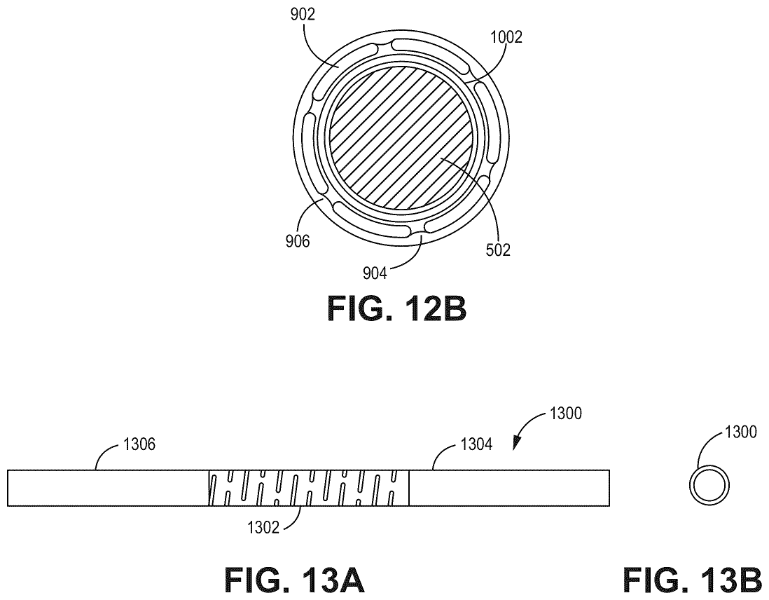

While the laser cut hypotube is very effective to improve flexibility, the surface of the cut hypotube can result in increased friction for objects moving across the surface. It has been discovered that friction between the laser cut hypotube and the corewire can be reduced during sliding of the corewire through the introduction of a coil between the corewire and the hypotube. Referring to FIGS. 10A and 10B, an inner coil 1002 is depicted that may be located between the corewire and laser cut hypotube to reduce friction when sliding corewire 502 within hypotube 902 to engage or disengage the fiber filter. Due to anticipated bends of the integrated guide structure when placed with its distal end in a cerebral artery, resistance to translation can result due to the capstan effect. Reducing the friction using the spring can reduce the capstan effect to provide for easier movement of corewire 502. Inner coil 1002 is fixed in position or has significantly constrained movement. FIG. 11 depicts an integrated device comprising an inner coil 1002 mounted proximally to the stake 702 over intermediate taper section of corewire 502 such that inner coil 1002 extends generally from uncut portion 1102 of the laser cut hypotube 902 to the stake 702. Polymer overcoat 904 may flow through laser cut slots 906 in the hypotube 902 and may hold inner coil 1002 in place so that the coil does not significantly translate laterally in either direction during use of the device. In an alternative embodiment shown in FIG. 12B, polymer overcoat 904 flows into, but not through laser cut slots 906. When polymer overcoat 904 does not engage inner coil 1002, inner coil 1002 in principle is not as constrained with respect to sliding within the laser cut hypotube, but various other constraints limit any movement of the inner coil, such as a change in inner diameter of the hypotube on the proximal side, and a stake or marker band on the distal side. Additional coatings or other structures may be used to reduce friction between the corewire 502 and laser cut hypotube 902. Marker bands can play multiple roles in the integrated guide structure. Generally, a fiber cartridge can have a marker band associated with each attachment feature to anchor the two ends of the fibers, a proximal marker band and a distal marker band. Visualization of the two marker bands coming together upon filter deployment and separating upon collapse of the filter provide real time visualization to facilitate confirmation of the fiber status. In addition, the fibers can be secured over the marker bands during formation of the filter cartridge. The proximal end of the filter cartridge is secured to the hypotube, and the corewire slides within the lumen of a proximal marker band. The distal end of the filter cartridge is secured to the corewire so that the distal end of the fiber cartridge moves with the corewire. A torque coupler at or near the proximal end of the fiber cartridge correspondingly preventing relative rotation of the hypotube and corewire allowing the maintenance of a twist in the fiber bundle. In general, marker bands may or may not play a significant role in the anchoring of the filter cartridge, but in the specific embodiments described herein, the marker bands are used to secure the filter cartridge at the respective ends. Marker band embodiments are described that provide a specific role in anchoring the fiber cartridge, and in one embodiment the proximal marker band also provides a torque coupling function. For assembly purposes, it can be desirable to separately form the filter cartridge incorporating a proximal marker band and a distal marker band for mounting on the corewire and appropriate attachment of the respective ends of the filter cartridge.

In the effort to improve flexibility around the distal end of the embolic protection device, it was discovered that one or more laser cut marker bands can also assist with the improvement in flexibility and consistency of the flexibility along the length of the device and through the fiber cartridge. In some embodiments, laser cutting the marker band may be inconsistent with combining the torque coupling function with the marker band, although conceivably compartmentalizing the marker band functions can provide domains with distinct functions with some laser cut portions. Embodiments of the filter cartridge involving positioning of laser cut marked bands along one or both ends of a fiber cartridge are described below. Furthermore, such laser cut marker bands can be useful in other devices with desired high flexibility, such as catheters or other devices, so the laser cut marker bands can have wider utility in which other devices may not have other functional purposes for the marker bands. The dimensions of the laser cut marker bands, diameter and length can be selected as suitable for the particular application.

Referring to FIGS. 13A and 13B, a laser cut marker band 1302 is depicted. Laser cut patterns and techniques may generally follow those described in regards in the laser cut hypotube above. The marker band may have laser cuts over almost the entire length of the marker band or over predetermined portions of the marker band. Generally, stability of the laser cut marker band suggests at least small uncut regions at the edges.

Referring to FIG. 13A, a laser cut marker band 1300 can have three sections, a central laser cut portion 1302, a first uncut portion 1304, and a second uncut portion 1306. If desired, a laser cut marker band can have more than three sections with different characteristics. While laser cut portion 1302 can have various shapes of metal removed, a spiral cut can be effectively provided to have desired flexibility. The relative extents of the cut and uncut sections can be selected for desirable mechanical properties. While FIG. 13A shows various portions of cut and uncut sections, these are not necessarily representative of a specific design for use. End view in FIG. 13B, shows the inner diameter and the outer diameter. The overall length and relative sizes of the regions along the laser cut marker bands can be selected to meet the objectives of the specific application. More specifics are described below in the context of use in a fiber cartridge.

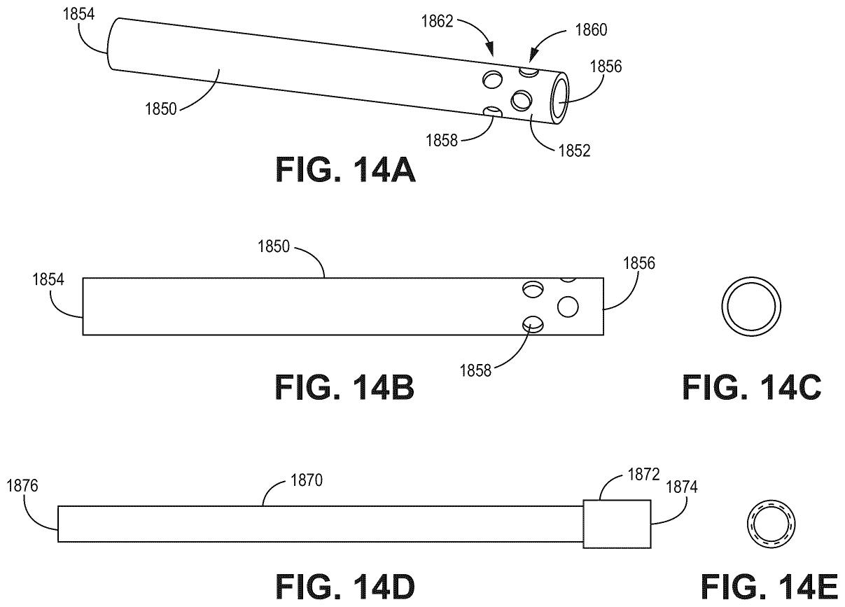

Referring to FIGS. 14A-E, alternative embodiments of marker bands are depicted for the distal marker band (FIGS. 14A-C) and proximal marker band (FIGS. 14D-E). Referring specifically to FIGS. 14A-C, a distal marker band 1850 is depicted with a fenestrated distal end 1852. In one embodiment, distal marker band 1850 may have an overall length from a distal opening 1854 to a proximal opening 1856 of about 2.5 inches. At fenestrated distal end 1852, distal marker band 1850 may have one or more holes 1858 cut through the wall to facilitate attachment to the corewire. The number, size and location of holes at the fenestrated region can be selected to facilitate their function and the placement of the holes allows access to them following assembly of the filter cartridge. FIGS. 14A-C depict one embodiment that provides for good attachment of the filter cartridge to the core wire while providing convenient manufacturing and handling. Alternative embodiments of the marker band can have different numbers of holes, different sized holes and different positioning of holes. As shown in FIGS. 14A-C, a first set of holes 1860 are cut such that each hole 1858 in first set 1860 has a center located about 0.17 inches from the proximal opening 1856. Each hole 1858 in first set of holes 1860 are generally equidistant from one other, being spaced with about 90 degrees of separation about distal marker band 1850. As shown in FIGS. 14A-C, a second set of holes 1862 are cut such that each hole 1858 has a center on a plane perpendicular to the axis of the marker band displaced about 0.17 inches from a plane through the centers of first set of holes 1860. Each hole 1858 in second set of holes 1862 are generally equidistant from one other, being spaced with about 90 degrees of separation about distal marker band 1850 and about 45 degrees from the orientations of first set of holes 1860. In a specific embodiment, each of the holes 1858 has a diameter of about 0.004 inches, although other reasonable diameters consistent with the other aspects of the marker band and its function can be used. In general, the holes can have dimeters from about 0.001 inches to about 0.01 inches. In a specific embodiment, the distal marker band 1850 has an inner diameter of about 0.00875 inches and an outer diameter of about 0.01055 inches. The inner diameter allows for mounting over the corewire with a correspondingly appropriate diameter, and the outer diameter follows according to the appropriate thickness of the marker band. As will be discussed in further detail below, when used as part of a filter cartridge, the holes 1858 allow reflowed polymer to pass through the wall of the distal marker band, creating a stronger bond with the filter cartridge.

Referring specifically to FIGS. 14D and 14E, a proximal marker band 1870 is depicted with a stepped up proximal portion 1872, which can provide for attachment to a hypotube. In a specific embodiment, proximal marker band 1870 may have a length of about 4.5 inches from a proximal opening 1874 to a distal opening 1876. In this embodiment, stepped up proximal portion 1872 may have a length of about 0.50 inches from the proximal opening 1874. The proximal marker band 1870 may have an outer diameter of about 0.01055 inches and in inner diameter of about 0.00875 inches. Stepped up proximal portion 1872 may have an outer diameter of about 0.014 inches, and this diameter can be selected to provide for desired attachment to a hypotube, which can be a laser cut hypotube.

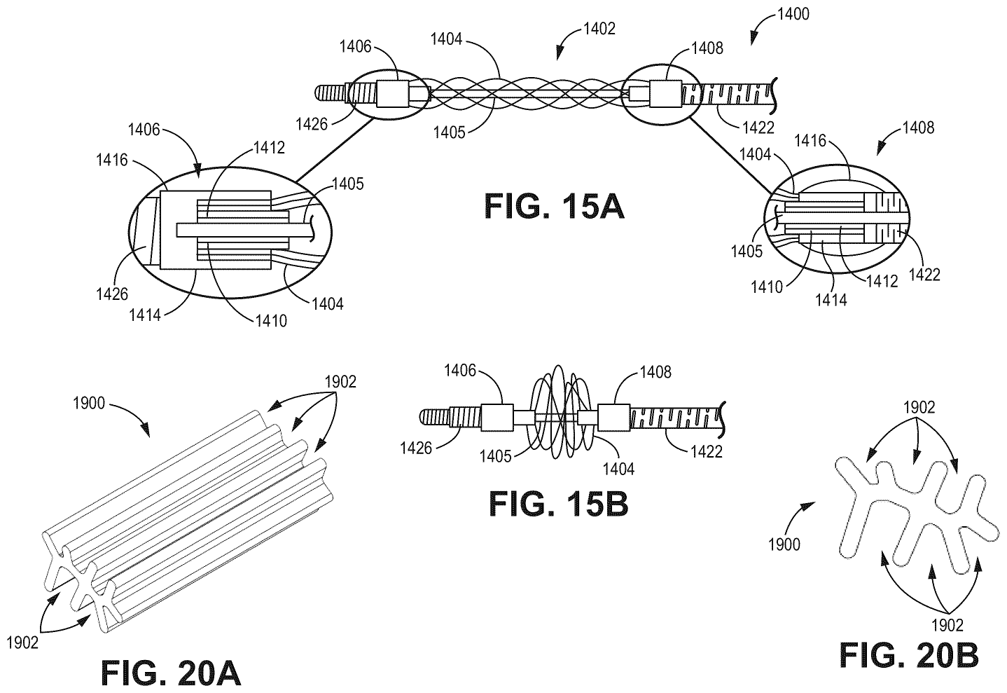

Referring to FIG. 15, in this fragmentary view, the embolism protection device 1400 comprises a filter structure 1402 in an undeployed configuration comprising a bundle of fibers 1404. FIG. 15A shows the filter in a low profile configuration for delivery into the deployment site, and FIG. 15B depicts the corresponding filter structure 1402 in a deployed configuration with bent fibers extending away from the corewire. Fibers within filter structure 1402 are arranged in a bundle of fibers 1404 and are generally polymer fibers that are stretched essentially straight for subsequent deployment in which the fibers are bent away from corewire 1405, as shown in FIG. 15B. Bundle of fibers 1404 can be twisted for improved performance as noted above, but twisting does not significantly change the overall profile of the undeployed bundle of fibers 1404. In some embodiments, the filter structure can comprise metal elements in addition to polymer fibers, as described further below. Fibers within bundle of fibers 1404 attached at respective ends at distal support 1406 and proximal support 1408. Proximal support 1408 is attached to and moves with hypotube 1422, and distal support 1406 is attached to and moves with corewire 1405. For filter deployment in the vessel, proximal support 1408 and distal support 1406 are brought closer together resulting in the bending of the fibers with the center of the fibers projecting outward relative to corewire 1405 to form a three-dimensional filtration matrix 1410.

Proximal attachment 1408 should be anchored relative to hypotube 1422. One or more anchoring modalities can be used. The one or more anchoring modalities should not interfere with the movement of the corewire relative to the hypotube to provide for actuation of the filter. Anchoring modalities can comprise a suitable connection of respective components, such as with adhesive, melt bonding, confining covers, welding, or other appropriate elements or processing. The materials for the anchoring modalities can be selected to maintain desirable flexibility while also maintaining the mechanical integrity of the assembled components in response to stresses expected during use of the device. A significant concern is the stability of the fiber cartridge and the fibers within the bundle. Two specific embodiments are discussed in some detail in the following.

Distal support 1406 and proximal support 1408 should provide secure anchoring of the elements of filter structure 1402, while maintaining reasonable flexibility to facilitate delivery and kink resistance. Distal support 1406 and proximal support 1408 generally can comprise an inner mounting tube 1410, radiopaque marker band 1412, adhesive 1414 and a cover 1416, such as a shrink wrap polymer, with polymer fibers 1404 fixed between them, as shown in the enlarged schematic sectional views in the balloon inserts associated with FIG. 15A. In some embodiments, all of these elements may not be used for either or both supports 1406, 1408, but the elements should be firmly secured to avoid any risk of detachment within a patient's vessel. In some embodiments for mounting filter structure 1402, distal support 1406 can be formed without an inner tube by securing marker band 1412 and/or bundle of fibers 1404 against corewire 1405, but the use of the inner polymer tube can provide production convenience for this element. In particular, a radiopaque marker band may function as an inner mounting tube. Proximal support 1408 should be attached to hypotube 1422 to provide for transitioning the fiber structure between deployed and undeployed states, but bundle of fibers 1404 cannot be mounted on the exterior of hypotube 1422 without making the diameter significantly larger. As shown in FIG. 14, proximal support 1408 can abut and overlap with hypotube 1422 and be appropriately attached to the fiber bundle and hypotube 1422 to provide desired security. An alternative embodiment is described below providing for a collar on the marker band with a stepped up diameter to provide for direct securing of the hypotube to the marker band. Use of a marker band within proximal support 1408 and distal support 1406 can provide desired imaging capability which can help to confirm deployment of the filter through the movement of the two marker bands toward each other through deployment of the filter, and potentially meeting when the filter is fully deployed.

Referring to the figure insert (FIG. 15A, left balloon) showing the sectional view of distal attachment 1406, corewire 1405 supports the structure with a distal coil 1426 mounted on the distal end of corewire 1405. To provide convenient mounting of a radiopaque marker band, the fibers can be secured over a marker band. With a laser cut marker band or in any case, it can be convenient to have polymer tube within the marker band. As shown in the figure insert, corewire 1405 extends to distal coil 1426, which are secured to each other at distal attachment 1406. Bundle of fibers 1404 are secured in distal attachment 1406 over radiopaque marker band 1410 (which can be laser cut), which in turn can be mounted over polymer tube 1412. Adhesive 1414 can be used to hold the fibers in place, although heat bonding of the fibers together can supplement or replace the adhesive. Cover 1416 can secure distal attachment 1406. Suitable materials for these elements are described further below. Cover 1416 can be a metal foil crimped over the structure, polymer shrink wrap, an adhered polymer sheet, combinations thereof, or the like. Generally, to resist the disengagement of an end of a fiber from supports 1406, 1408, polymer fibers can be heat bonded to each other if the polymers are made from a suitable material and the other elements are stable with the temperatures involved, which may replace adhesive 1416 with corresponding appropriate attachment of distal coil 1426 to corewire 1405, such as with welding or solder. Cover 1416 covers the exterior of the secured ends of the fiber cartridge and can help to connect with adjacent structures. Referring to the insert for the expanded cross section of proximal support 1408, a similar structure is shown as used for distal support 1406. Proximal support 1408 involves hypotube 1422 rather than distal coil 1426. Fibers from bundle of fibers 1404 are attached over radiopaque marker band 1410 (which may be laser cut), which is placed over polymer tube 1412.

For proximal support 1408, corewire 1405 can slide within polymer tube 1412. Adhesive 1414 can help to secure fibers, hypotube 1422, radiopaque marker band 1410, polymer tube 1412 together. Polymer tube 1412 is generally placed between radiopaque cut marker band 1410 and corewire 1405 at proximal support 1408 to facilitate sliding of the corewire relative to the marker band and to avoid adhesive connecting the marker band to the corewire. Adhesive may penetrate through the laser cuts of the marker band to adhere the marker band to the polymer tube at proximal support 1408 and to the corewire or polymer tube at distal support 1406. Cover 1416 can provide connection or assist with connection of the fiber cartridge with hypotube 1422.

It may be desirable to use more flexible adhesives to assist with maintaining good flexibility through the attachment regions of the device. Suitable more flexible adhesives include, for example, silicon adhesives. Medical device grade silicon adhesives are available from medical adhesive brand lines, for example, MasterBond®, Hankel Loctite® (e.g., 5055 or 5056), and within these brands adhesives can be selected to provide reasonable values of elasticity. Silicon adhesives may bond well to metal, so a marker band may be used without a polymer jacket or coating. More traditional, less flexible medical grade adhesives would include cyanoacrylates (e.g., Hankel Loctite® 4311), epoxies, acrylated urethanes (e.g., Dymax MD® Medical Device Adhesives 203A-CHT-F-T or 1128A-M) and the like, although some specific adhesive products based on these chemistries may be more elastic. A combination of adhesives could be used to leverage flexibility over the majority of an attachment and security of a stronger adhesive at stress points, and blends of adhesives may also provide desired properties.

To provide improved flexibility through the fiber cartridge, attachments 1406, 1408 can be accordingly designed to be shorter with respect to extent along the corewire and more flexible. In some embodiments, flexibility can be enhanced by using a laser cut marker band. An embodiment of a laser cut marker band is shown in FIG. 13. As noted above, laser cut marker bands can be used for other purposes effectively in various medical devices, such as catheters of various designs, with appropriate selection of dimensions for the particular use. For incorporation into a filter cartridge, a laser cut marker band can have a length from about 0.1 in. to about 0.5 in and in. further embodiments from about 0.125 in. to about 0.4 on. Generally, at least about 20% of the length is laser cut, and positioning of the laser cut section along the length can be selected based on the configuration in the attachment section. The inner diameter should provide for passage of the corewire and optionally, polymer tube, that can be placed between the laser cut marker band and the corewire to provide for smooth movement of the corewire for a proximal laser cut marker band. The corewire can have a tapered diameter at the filter cartridge. For this application, the inner diameter of radiopaque marker band 1410 can be from about 0.006 in. to about 0.015 in. and in further embodiments from about 0.0075 in. to about 0.0135 in., and the outer diameter can be from about 0.008 in. to about 0.019 in. and in further embodiments from about 0.009 in. to about 0.175 in. A person of ordinary skill in the art will recognize that additional ranges of marker band dimensions within the explicit ranges above are contemplated and are within the present disclosure.

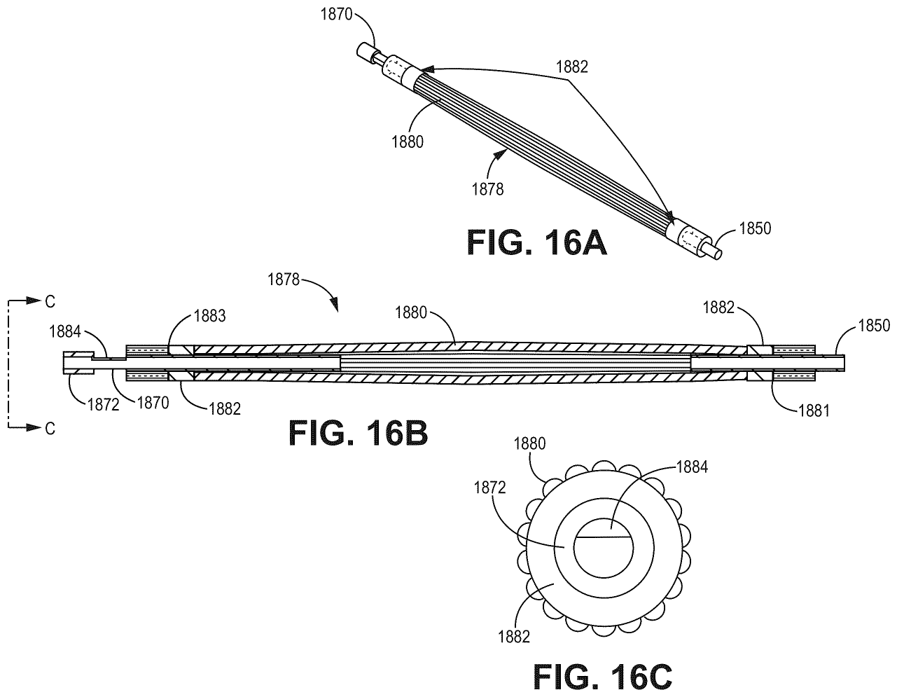

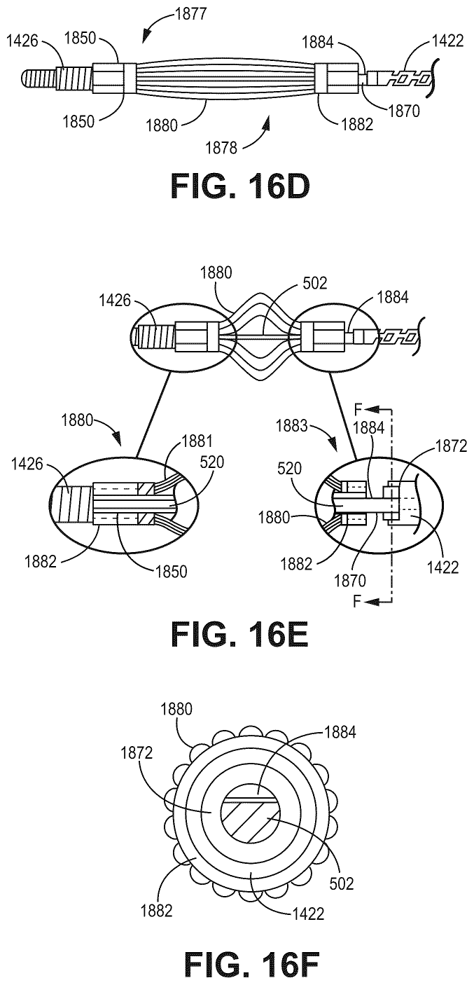

As noted above, in one embodiment of particular interest, a torque coupler component is incorporated into a proximal marker band. In these embodiments, the proximal marker band comprises three regions to separately provide securing to the hypotube, providing a torque coupler and attachment of the proximal end of the fibers with extending portion of the marker band in the interior of the filter cartridge being configured to interact with a distal marker band. In one embodiment of particular interest, a distal marker band can comprise fenestrations near its distal end to enhance securing of the marker band and distal end of the fiber cartridge to the corewire, although alternative embodiments can comprise only one or the other of these marker band designs. The marker band embodiments are depicted in FIGS. 14A-C. This embodiment of the filter cartridge is depicted in FIGS. 16A-C separated from the corewire.

Referring to FIGS. 16A-C, a specific embodiment of a filter device 1878 is depicted with a distal marker band 1850 and a proximal marker band 1870. As seen in the cross sectional view in FIG. 16B, fibers 1880 extend over a portion of the marker bands 1850, 1870. Distal and proximal ends of fibers 1880 are fixed in place about the marker bands. In some embodiments, polymer 1882, such as a PET shrink wrap, may be used to secure the fibers. In some embodiments, other adhesives or welds may be used to secure the fibers to the marker bands. As noted above, on the distal marker band 1850, the polymer may flow through the fenestrations in the proximal end to more securely adhere the fibers to the marker band.

The proximal marker band 1870 may have a crimped portion 1884 between the stepped up proximal portion 1872 and the polymer 1882. The crimped portion 1884 may act as a torque coupler preventing the corewire from rotating freely within the marker band or filter cartridge. In some embodiments, the crimped portion 1884 may be used in combination with the stake 702, as described above, as a secondary torque coupler. In some embodiments, the crimped portion 1884 may be used as an alternative torque coupler.

Referring to FIGS. 16D, in this fragmentary view, the embolism protection device 1877 comprises filter structure 1878 connected with hypotube 1422 and corewire 520, which is not visible in this view. Filter structure 1878 is in an undeployed configuration and comprises a bundle of fibers 1882. FIG. 16D shows the filter in a low profile configuration for delivery into the deployment site, and FIG. 16E depicts the corresponding filter structure 1878 in a deployed configuration with bent fibers extending away from the corewire 520. Fibers within filter structure 1878 are arranged in a bundle of fibers 1880 and are generally polymer fibers that are stretched essentially straight for subsequent deployment in which the fibers are bent away from corewire 520, as shown in FIG. 16E. Bundle of fibers 1880 can be twisted for improved performance as noted above, but twisting does not significantly change the overall profile of the undeployed bundle of fibers 1880. In some embodiments, the filter structure can comprise metal elements in addition to polymer fibers, as described further below. Fibers within bundle of fibers 1880 attached at respective ends at distal support 1881 and proximal support 1883. Proximal support 1883 is attached to and moves with hypotube 1422, and distal support 1881 is attached to and moves with corewire 520. For filter deployment in the vessel, proximal support 1883 and distal support 1881 are brought closer together resulting in the bending of the fibers with the center of the fibers projecting outward relative to corewire 502 to form a three-dimensional filtration matrix 1410.

Proximal attachment 1883 should be anchored relative to hypotube 1422. One or more anchoring modalities can be used. The one or more anchoring modalities should not interfere with the movement of the corewire relative to the hypotube to provide for actuation of the filter. Anchoring modalities can comprise a suitable connection of respective components, such as with adhesive, melt bonding, confining covers, welding, or other appropriate elements. The materials for the anchoring modalities can be selected to maintain desirable flexibility while also maintaining the mechanical integrity of the assembled components in response to stresses expected during use of the device. A significant concern is the stability of the fiber cartridge and the fibers within the bundle. Welding of proximal attachment 1883 to proximal marker band 1870 can be desirable due to the ability to form a strong bond without addition of another material, although adhesive may be placed over the welded structure.

Distal support 1881 and proximal support 1883 should provide secure anchoring of the elements of filter structure 1878, while maintaining reasonable flexibility to facilitate delivery and kink resistance, while securing the ends of the fibers. Distal support 1881 can generally comprise distal marker band 1850, adhesive and a cover 1882, such as a shrink wrap polymer, with polymer fibers 1880 fixed at their distal ends over marker band 1850, as shown in the enlarged schematic sectional views in the balloon inserts associated with FIG. 16E. As noted above, distal marker band 1850 may include one or more fenestrations polymer, such as adhesive, to flow through and enhance anchoring of the elements to corewire 520. Proximal support 1883 generally can comprise proximal marker band 1870, adhesive and a cover 1882, such as a shrink wrap polymer, with polymer fibers 1880 fixed between them. In some embodiments, all of these elements may not be used for either or both supports 1881, 1883, but the elements should be firmly secured to avoid any risk of detachment within a patient's vessel. As shown in FIG. 16E, hypotube 1422 can abut and overlap proximal support 1883. In a specific embodiment, the stepped up proximal portion 1872 on the proximal marker band 1870 provides for direct securing of the hypotube to the marker band. Use of a radiopaque marker bands within proximal support 1883 and distal support 1881 can provide desired imaging capability which can help to confirm deployment of the filter through the movement of the two marker bands toward each other through deployment of the filter, and potentially meeting when the filter is fully deployed.

Referring to the figure insert (FIG. 16E, left balloon) showing the sectional view of distal attachment 1881, corewire 520 supports the structure with a distal coil 1426 mounted on the distal end of corewire 520. To provide convenient mounting of a radiopaque marker band, the fibers can be secured over a marker band, such as distal marker band 1850. As shown in the figure insert, corewire 520 extends to distal coil 1426, which are secured to each other at distal attachment 1881. Bundle of fibers 1880 are secured in distal attachment 1881 distal marker band 1850. Adhesive can be used to hold the fibers in place, although heat bonding of the fibers together can supplement or replace the adhesive. Cover 1882 can secure distal attachment 1881. Suitable materials for these elements are described further below. Cover 1881 can be a metal foil crimped over the structure, polymer shrink wrap, an adhered polymer sheet, combinations thereof, or the like. Fenestrations in distal marker band 1850 may allow adhesive to flow through for improved bonding of the structure. Generally, to resist the disengagement of an end of a fiber from supports 1881, 1883, polymer fibers can be heat bonded to each other if the polymers are made from a suitable material and the other elements are stable with the temperatures involved, which may replace or supplement adhesive. Adhesive may also be used for appropriate attachment of distal coil 1426 to corewire 502, although attachment of the distal coil may also involve welding or solder. Cover 1882 covers the exterior of the secured ends of the fiber cartridge and can help to connect with adjacent structures. Referring to the insert for the expanded cross section of proximal support 1883, a similar structure (in a mirrored configuration) is shown as used for distal support 1881, although the proximal support comprises an element for torque coupling and has a different structure for attachment to connected structures. Proximal support 1883 involves connection with hypotube 1422 rather than distal coil 1426, and corewire 520 is secured to distal support 1881 while corewire 520 slides relative to proximal support 1883. Fibers from bundle of fibers 1880 are attached over proximal marker band 1870. For proximal support 1883, corewire 502 can slide within polymer proximal marker band 1870. Adhesive can help to secure fibers, hypotube 1422, and proximal marker band 1870 together. Adhesive may penetrate through the fenestrations of the distal marker band 1850 to adhere the marker band at distal support 1881 to the corewire 502.

Suitable adhesives are discussed above.

As noted above, in the embodiment of FIG. 16, a torque coupler component is incorporated into a proximal marker band. Proximal marker band 1870 may have a crushed or crimped portion 1884. In some embodiments, crimped portion 1870 may be generally adjacent to stepped up proximal portion 1872. Crimped portion 1870 generally corresponds to flattened section 508 of corewire 502, as seen in the sectional view of FIG. 16F. Accordingly, corewire 502 may slide laterally along the length of the device a sufficient distance to deploy or un-deploy filter structure 1878.

In some embodiments, the fiber cartridge can comprise additional elements within the fiber bundle to provide particular features to the deployed filter. For example, the fiber bundle can comprise metal wires with diameters on the same order as the polymer fibers. In some embodiments, to avoid potentially undesirable contact with the vessel wall, the metal wires can be loaded into the bundle closer to the corewire relative to the majority of the polymer fibers so that they likely can be more central in the deployed filter matrix. The metal wires can be formed from shape memory metals such that the wires can take a contorted shape if desired upon deployment.

Wires with a controlled shape similarly can be configured to avoid vessel wall contact after deployment.

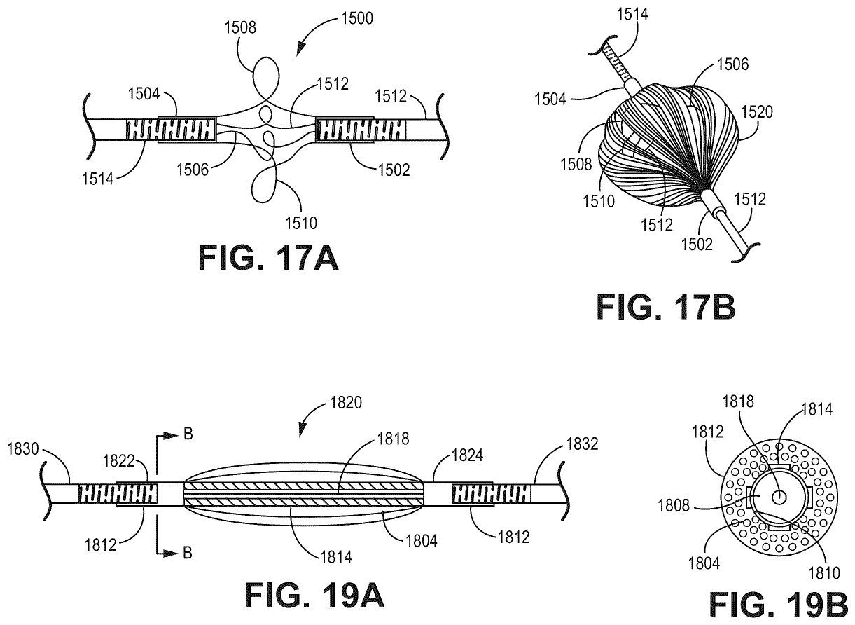

Such configurations of metal wires in fiber-based filter matrices are shown in FIG. 17. Referring to FIG. 17, embolism protection device 1500 is depicted with polymer fibers removed to show metal wires in a partially deployed configuration without obstruction by the polymer fibers. Embolism protection device 1500 comprises proximal support structure 1502, distal support structure 1504 and four unwoven metal elements 1506, 1508, 1510, 1512 designed to extend into a curved structure. Proximal support structure 1502 connects with hypotube 1512, and distal support structure connects with distal coil 1514. Metal elements 1506, 1508, 1510, 1512 can be made of a radiopaque metal, such as platinum, to further provide information on filter deployment. While shown with 4 metal wires in FIG. 17, a greater or lesser number of metal elements can be used, such as two metal elements, five metal elements, six metal elements, seven metal elements, eight metal elements or greater than 8 metal elements. The metal wires can have comparable diameters as polymer fibers. A depiction of embolism protection device 1500 is shown in FIG. 18 in which extended metal elements 1506, 1508, 1510, 1512 are cushioned by the polymer fibers 1520 that extend radially outward to the extent provided by the constrained ends of the fibers. A fiber filter with shape memory metal wires is described further in the '386 patent cited above. Referring to FIG. 19, depicted filter cartridge 1820 comprises distal support 1822, proximal support 1824, metal elements 1814, and polymer fibers 1804. Distal support 1822 and proximal support 1824 generally can comprise an inner mounting tube 1808, radiopaque marker band 1812, and a cover 1822, such as a shrink wrap polymer, with polymer fibers 1804 fixed between them, as shown in the enlarged sectional view in FIG. 19B. In some embodiments, metal elements 1814 can act as struts to provide mechanical stability to the deployed filter. In some embodiments, all of these elements may not be used, but the elements should be firmly secured to avoid any risk of detachment within a patient's vessel. In some embodiments, distal support 1822 and proximal support 1824 can be designed analogously to supports 1406, 1408 of FIG. 15. Distal support 1822 is connected to distal coil 1830, and proximal support 1824 is connected to hypotube 1832. Corewire 1818 extends through the filter cartridge analogously to the embodiments above in FIGS. 15-18.

The number of fibers in the bundle generally depends on the desired degree of filtration as well as the thickness of the fibers and the acceptable overall thickness of the fiber bundle. As described below, surface capillary fibers have been found to contribute to desirable emboli capture, which may be attributable to their high surface area. In other embodiments, round polymer fibers can be used while still providing desirable filtration performance. For delivery into small vessels, it can be desirable to have a smaller dimension for the undeployed fiber cartridge, that then correlates with fewer fibers or thinner wires. An embodiment of a filter cartridge is presented below in which added length of the filter cartridge can be translated into multiple of deployed filter thickness.

Individual fibers can be organized into yarns with each strand of yarn then being incorporated into a fiber bundle, although alternatively individual fibers can be assembled into the fiber cartridge. In general, the number of strands of yarn in a fiber bundle can range from at least 10 strands, in further embodiments from 25 strands to 1,000 strands, in other embodiments from 50 strands to 750 strands, and in further embodiments form about 75 strands to about 500 strands. The number of strands generally depends on the thickness of the strands, which in turn depends on the number of fibers in a strand and the thickness of a fiber, as discussed further below.

In some embodiments, each fiber bundle can have from about 25 fibers to about 2500 fibers, in some embodiments from about 50 fibers to about 1500 fibers and in further embodiments from about 75 fibers to about 1000 fibers. For use in cerebral vessels, it can be desirable for the undeployed filter cartridge to have a maximum diameter of no more than about 0.050 in (1.25 mm), in further embodiments from about 0.010 in (0.25 mm) to about 0.040 in (1.0 mm) and in other embodiments from about 0.020 in (0.5 mm) to about 0.035 in (0.889 mm). If used in other vessels, larger diameters may be acceptable, such as two or three times theses values. A person of ordinary skill in the art will recognize that additional ranges of fiber and fiber cartridge dimensions within the explicit ranges above are contemplated and are within the present disclosure.

The length of the fibers can be selected based on the size of the corresponding vessel. When deployed, the centers of the fibers are projected across the lumen of the vessel. Thus, the unconstrained length of the fibers between attachment structures 1406, 1408 should be at least double the radius of the vessel. In some embodiments relating to the use of a plurality of fibers to expand within the lumen of a patient's vessel, it is generally appropriate to use fibers that have a length from about 2.2 to about 10 times the vessel radius, in some embodiments from about 2.4 to about 5 times the vessel radius and in further embodiments from about 2.6 to about 4 times the vessel radius. For placement in a human vessel, the fibers generally have a length from about 0.25 mm to about 25 mm, in other embodiments from about 0.5 mm to about 20 mm, and in further embodiments from about 1 mm to about 15 mm. With respect to lengths, for use in cerebral vessels, the unconstrained lengths of the fibers can be from about 2 mm to about 10 mm. For other vessels, the fiber lengths can be longer. The anchored lengths of the fibers on each end depends on the design of the attachment element, as described above. A person of ordinary skill in the art will recognize that additional ranges of fiber numbers and fiber length within the explicit ranges are contemplated and are within the present disclosure.

As used herein, SCF fibers (Surface Capillary Fibers) refer broadly to fibers having channels or capillaries along the surface running generally along the length of the fiber or a portion thereof. Fibers have their usual meaning as structures with a length that is significantly larger than the dimensions along a cross section perpendicular to the length. The capillaries can run along substantially the entire length or a fraction thereof. Due to the presence of the capillaries, a cross section through the fiber at the capillary (ies) has a shape with an edge having changing curvatures. The fibers are generally organized into yarns, which form the fibers for assembling the fiber cartridge.

SCF fibers for use in the medical devices are generally formed from biocompatible polymers. SCF fibers can be fabricated from synthetic polymers as well as purified biological polymers and combinations thereof. Suitable synthetic polymers include, for example, polyamides (e.g., nylon), polyesters (e.g., polyethylene teraphthalate), polyacetals/polyketals, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Based on desirable properties and experience in the medical device field, suitable synthetic polymers include, in particular, polyether ether ketones, polyacetals, polyamides (e.g., nylons), polyurethanes, polytetrafluoroethylene, polyester teraphthalate, polycarbonates, polysulfone and copolymers and mixtures thereof.

Bioresorbable synthetic polymers can also be used such as dextran, hydroxyethyl starch, derivatives of gelatin, polyvinylpyrrolidone, polyvinyl alcohol, poly [N-(2-hydroxypropyl) methacrylamide], poly(hydroxy acids), poly (epsilon-caprolactone), polylactic acid, polyglycolic acid, poly(dimethyl glycolic acid), poly(hydroxy butyrate), and similar copolymers. Based on experience in the medical field, suitable resorbable polymers include, in particular, polylactic acid, polyglycolic acid, and copolymers and mixtures thereof.

Appropriate polymers also include biological polymers. Biological polymers can be naturally occurring or produced in vitro by fermentation and the like. Suitable biological polymers include, without limitation, collagen, elastin, silk, keratin, gelatin, polyamino acids, cat gut sutures, polysaccharides (e.g., cellulose and starch) and mixtures thereof. Biological polymers generally are bioresorbable. Purified biological polymers can be appropriately formed into a polymer material for further processing into fibers.

The properties of the surface channels and the corresponding cross-section of the fiber generally depends on the process used to form the fibers. Fiber with fairly complex surface channel geometry are described in U.S. Pat. No. 5,972,505 to Phillips et al., entitled “Fibers Capable Of Spontaneously Transporting Fluids,” incorporated herein by reference. A further approach for forming a fiber with surface capillaries is described in U.S. Pat. No. 5,200,248 to Thompson et al. (hereinafter the '248 patent), entitled “Open Capillary Channel Structures, Improved Process For Making Capillary Channel Structures, And Extrusion Die For Use Therein,” incorporated herein by reference. Fibers based on the '248 patent can be particularly useful for filter applications. The fiber development efforts in the '505 and '248 patents were extended in U.S. Pat. No. 6,103,376 to Phillips et al., “Bundles of Fibers Useful for Moving Liquids at High Fluxes and Acquisition/Distribution Structures That use the Bundles,” incorporated herein by reference. Bundles of fibers based on technologies under these patents are available as 4DG Fibers from Fiber Innovation Technology, Inc. Representative views of 4DG fibers 1900 are shown in FIGS. 20A (perspective view) and 20B (end view). 4DG fibers 1900 have surface channels 1902 running along their length.

Surface capillary fibers or other fibers can be extruded or otherwise formed with polymer loaded with a radiopaque material, such as barium titanate, platinum particles or the like, to render the fibers more visible under x-ray imaging. The use of radiopaque fibers for embolic protection is described in U.S. Pat. No. 8,052,714 to Galdonik et al., entitled “Radiopaque Fibers and Filtration Matrices,” incorporated herein by reference.

As with the fiber length, the thickness of the fibers can be selected appropriately for the particular use of the fiber as well as any practical constraints of the supplier. Fiber thickness can be measures in several ways. As described in the previous paragraph, the radius of the fiber can be roughly estimated from the assumption of a circular cross section. Alternatively, one can define an average diameter by taking an average cross section and then averaging the length of segments through the center of the cross section that intersect the circumference of the cross section. Also, calipers can be used to measure thickness, which can be averaged to obtain a value of the diameter. These various approaches at estimating the radius or diameter generally give values of roughly the same magnitude. Also, in the fiber field, a pragmatic way has been developed to characterize fiber thickness without the need to resort to magnification of the fibers. Thus, fiber thickness can be measured in units of denier. Deniers correspond to the number of grams per 9,000 meters of yarn with a larger value corresponding to a thicker fiber. In some embodiments, suitable fibers have diameters from 50 microns to about 5 millimeter, in further embodiments from about 100 microns to about 2 millimeters, and in additional embodiments from about 150 microns to about 1 millimeter. As measured in denier, SCF fibers can have sizes ranging from about 0.1 denier to about 100 denier in size, in additional embodiments from about 0.5 denier to about 75 denier, and in some embodiments from about 1.0 denier to about 50 denier. A person of ordinary skill in the art will recognize that additional ranges of fiber thickness in diameter measurements or in denier are contemplated and are within the present disclosure.

Further characterization of the fibers can borrow from the approaches outlined in the '248 patent. In particular, the overall capillary sizes can be characterized. In some embodiments of interest, the fibers have a specific capillary volume of at least about 0.5 cc/g, in other embodiments at least about 1.0 cc/g, in further embodiments at least about 2.0 cc/g and in additional embodiments at least about 3.0 cc/g. Also, the specific capillary surface area can be at least about 500 cm2/g, in some embodiments at least about 1000 cm2/g, in further embodiments at least about 2000 cm2/g, and in other embodiments at least about 3000 cm2/g. A person of ordinary skill in the art will recognize that additional ranges of capillary volumes and capillary surface areas are contemplated and are within the present disclosure. Test methods for evaluating the specific capillary volume, the specific surface capillary area and the adhesion tension are described in detail in the '248 patent, which is incorporated herein by reference for the explicit description of the determination of these values.] In some embodiments, the fibers have a tensile strength of about 10 grams to about 20 grams, which can be measured based on an ASTM standard.

The above polymers can also be made in non-capillary fibers. Further, ePTFE, sintered PTFE, and spun nylon could also be used. These fibers can be combined with metal braided, woven, or laser cut structures. The fibers can be woven through the metal patters, placed on the inside, on the outside, or some combination thereof.

The particular preparation processes for the fibers can lead to significantly improved uniformity of the performance of the embolism protection device. In particular, the fibers can be twisted within the fiber bundle mounted for deployment. The torque coupler can effectively maintain the twist of the fiber cartridge. While any degree of twist can be desirable, twist can be applied to the fiber bundle of at least about 5 degrees and in further embodiments from about 180 degrees to about 360 degrees. Furthermore, multiple rotations, for example, about 360 degrees to about 1080 degrees, can further act to increase the density of fibers and may be advantageous. A person of ordinary skill in the art will recognize that additional ranges of twist within the specific ranges above are contemplated and are within the present disclosure. The twist can be applied by fastening one end of the fiber bundle, applying the twist and fastening the other end of the fiber bundle. A suitable torque coupler can facilitate the application and maintenance of the twist to the fibers since the corewire does not rotate due to tension in the fibers. With the application of a suitable twist, the embolism protection device has been observed to perform with essentially uniform performance. The effects of twisting a fiber based filter device is described in U.S. Pat. No. 7,988,705 to Galdonik et al., entitled “Stecrable Device Having a Corewire Within a Tube and Combination With a Functional Medical Component,” incorporated herein by reference.

Referring to FIG. 1, distal end 120 of the embolic protection device generally comprises a distal coil 1202, such as shown in one embodiment in FIGS. 18A and 18B. In the embodiment of FIG. 18, distal coil 1202 comprises a larger diameter proximal section 1204, a smaller diameter distal section 1206 and a rounded tip 1208, which can be a solder ball or other suitable structure to provide a non-traumatic tip. An end view if distal coil 1202 is shown in FIG. 18B. Distal coil generally is attached to the corewire at a distal support that connects the corewire, filter cartridge and distal coil, as described above in the context of FIG. 14.

As suggested above, the presentation of a low filter diameter is desirable to maintain flexibility and to facilitate entry into small blood vessels and advancing past a clot. Having a lower diameter for the filter cartridge restricts the number and/or diameter of the fibers, which has a corresponding effect on the deployed filter matrix. On the other hand, while length may not freely extendable, the length may have different constraints providing a tradeoff with respect to the number of fibers and the length of the undeployed filter cartridge. By using a series of deployable filter matrices in a compound filter, the filly deployed filter may have a double thickness of filtration matrix, which may provide excellent embolic protection with a fewer number of fibers in the respective bundles.

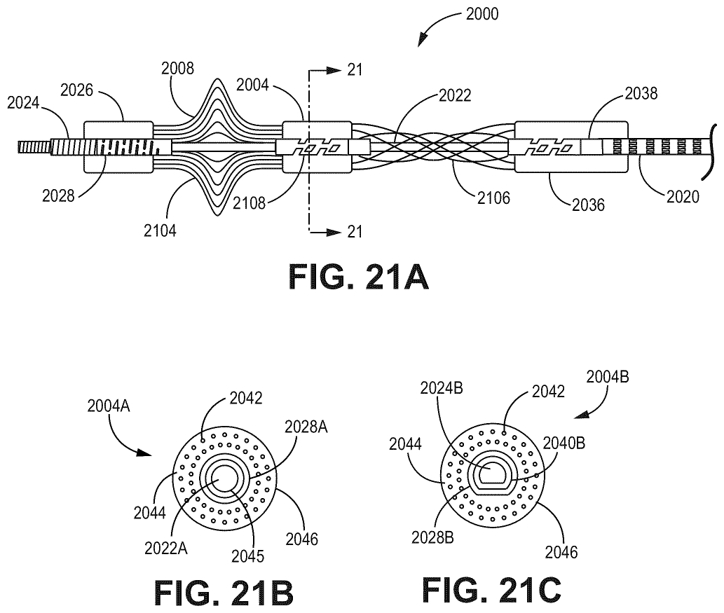

Referring to FIG. 21A, an embodiment of an embolism protection device 2000 is depicted that comprises a compound fiber cartridge 2002 that has a central attachment 2004 that separates a proximal filter component 2006 and distal filter component 2008. Distal filter component 2006 is depicted in a partially deployed configuration, and proximal fiber component 2008 is depicted in a delivery or narrow profile configuration. Embolism protection device 2000 generally comprises hypotube 2020, which may be laser cut for flexibility, corewire 2022, and distal coil 2024. Distal attachment 2026 secures distal filter component 2008 and distal coil 2024 to corewire 2022 at radiopaque marker band 2028, which can be laser cut, and radiopaque marker band 2028 can be placed over a polymer tube (not shown). Similarly, proximal attachment 2036 secures proximal filter component 2006 to hypotube 2020 at radiopaque marker band 2038, which can be laser cut, and radiopaque marker band 2038 can be placed over a polymer tube to facilitate sliding of corewire 2022. Distal attachment 2026 and proximal attachment 2036 can comprise adhesive and/or a cover to secure the elements together as described above in the context of comparable attachments 1406 and 1408 above in FIG. 15.

Central attachment 2004 slides over corewire 2022 in conjunction with deployment of the filter since it gets closer to hypotube 2020 to deploy proximal filter component 2006 and closer to distal coil 2024 to deploy distal filter component 2008. Central attachment 2004 may or may not be able to rotate around distal coil 2024. As noted above, it can be desirable to twist the fibers and lock the relative rotation of the corewire and hypotube. If central attachment 2004 freely rotates, central attachment 2004 can rotate with the fibers in the twist. If central attachment 2004 has a fixed orientation around corewire 2022, then the twists of proximal filter component 2006 and distal filter component 2008 can be separately set. Referring to FIG. 21B, a sectional view is shown of a first embodiment of central attachment 2004A, with corewire 2022A, polymer tube 2040A, radiopaque marker band 2028A, secured fiber segments 2042, adhesive 2044 and cover 2046. As shown in FIG. 21B, tapered corewire has a circular cross section and polymer tube 2040A has a similar circular cross section to allow for relative rotation, although the corewire could have an appropriate noncircular cross section while maintaining rotational freedom if polymer tube has a circular cross section. Referring to the embodiment in FIG. 21C, the sectional view depicts corewire 2022B, polymer tube 2040B and radiopaque marker band 2028B with non-circular cross sections the restrict any relative rotation, and secured fiber segments 2042, adhesive 2044 and cover 2046 comparable to FIG. 21B accounting for placement around non-circular central components. Fibers in proximal filter component 2006 and distal filter component 2008 can be common long fibers appropriately anchored in central attachment 2004, or two sets of distinct fibers commonly secured at central attachment 2004. While either design can function appropriately, having common long fibers can avoid any concerns over pulling out the fiber ends from central attachment 2004, which may allow for a shorter length of central attachment 2004.

When deployed by pulling the corewire proximal relative to the hypotube, both proximal filter component 2006 and distal component 2008 deploy. As shown in FIG. 21A, the filter components may or may not simultaneously deploy. The relative deployment rate may depend on friction and other balance of forces during deployment, but the ultimate deployed filter should not matter on the particular deployment order. In any case, the deployed filter has an effective filtration matrix formed essentially as if double the number of fibers were used such that a thinner filter cartridge can be used without compromising the filter efficacy. Similarly, a triple filter cartridge or higher order filter cartridge can be similarly used, up to some practical limit.

For assembly of the embolic protection device, the corewire should be inserted into the hypotube with proper alignment of the corewire structure at the desired position along the length of the hypotube. This alignment then provides an appropriate length of the corewire extending from the respective end of the hypotube to allow for relative movement of the corewire at the proximal end and attachment of the filter cartridge and distal coil at the distal end. Additional components can be slid over the corewire's distal end. In particular, an inner coil can be inserted to line interior of the laser cut hypotube, and a stake for torque coupling can then be placed following insertion of the inner coil. With these components in place, the thermoplastic elastomer can be reflowed over the exterior of the laser cut hypotube to anchor the stake and provide for rotationally fixing the corewire within the hypotube. Once the stake is immobilized in the hypotube, the inner coil is blocked from exiting the hypotube.

The filter cartridge can be assembled with respect to fixing fibers to the radiopaque marker band/polymer tube either mounted on the corewire, or separately from the corewire, or using one approach for one end of the filter cartridge and the other approach for the other end of the filter cartridge. The assembly and mounting of the filter cartridge should provide to placement and maintenance of the twist of the filter cartridge. Generally, at least the cover securing the filter cartridge to the hypotube on the proximal end and the distal coil on the distal end is performed with the filter cartridge in final position. The assembly process should provide for organizing the fibers roughly evenly around the circumference of the attachment and then fixing the fibers with heat bonding, adhesive, a combination thereof of the like. If the assembly is performed on the corewire, the attachment of the fibers can be done simultaneously with the attachment to the adjacent structural elements, the hypotube on the proximal end and the corewire/distal coil on the distal end. One end can be fully secured to provide for twisting the fiber cartridge and then the other end can be secured to the adjacent structure, and in principle either side can be secured first.

Control of filter deployment and disengagement involves relative movement of the corewire and hypotube. While this can be done manually, appropriate tools can facilitate this process and provide for controlled transitions that account for the expected amounts of relative motion. Actuation tools developed for the predecessor FiberNet™ device can be adapted for use with the present device. Convenient devices provide for locking of the ends of the corewire and hypotube and providing levers to move these relative to each other while supporting the fragile corewire to avoid kinking. To provide for curving the laser cut hypotube, it can be desirable to advance the corewire in a relative distal direction from the position with the fiber cartridge in an undeployed neutral position. The actuators can be designed with a range of movement of the corewire relative to the hypotube to provide for curvature of the hypotube as well as deployment and collapse of the deployed filter.

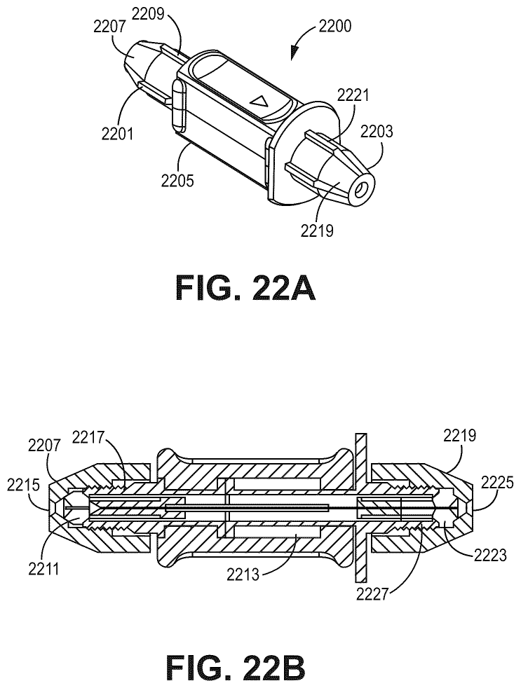

A vice grip and actuating assembly 2200 having a distal portion 2201, proximal portion 2203, and central portion 2205 is shown in FIGS. 22A and 22B. Distal portion 2201 is configured to clamp onto the corewire and proximal portion 2203 clamps onto the hypotube. As seen in the sectional view of FIG. 22B, central portion 2205 contains an interior cavity 2213 such that proximal portion 2203 may have a limited range to slide axially with respect to distal portion 2203. In embodiments, interior cavity 2213 has a length between 8 and 15 mm, and this can be selected based on the dimensions of the filter cartridge. With the distal portion 2201 clamped onto the hypotube and the proximal portion 911 clamped onto the corewire, moving distal portion 901 and proximal portion 2211 with respect to one another in effect translates the corewire and hypotube with respect to one another. As explained in more detail above, sliding the corewire with respect to the hypotube actuates the filter element, causing it to expand or retract.

Distal head 2207 may have one or more ribs 2209 and surrounds distal collet assembly 2211. Ribs 2211 may make it easier to turn distal head 2207 in order to either retain or release the control wire. Distal collet assembly 2211 has a thru hole 2215 configured to receive control wire. When control wire is inserted into thru hole 2215, rotating head 2207 about threads 2217 in a first direction causes collet 2211 to clamp down on control wire in a vise like grip, and rotating head 2207 in an opposite direction causes collet 911 to release control wire. When control wire is secured by collet 2211, collet assembly 2200 may be manipulated to exert control over the control wire. For example, twisting collet holder control assembly 2200 may place torque on a control wire.

Proximal head 2219 may have one or more ribs 2221 and surrounds distal collet assembly 923. Ribs 911 may make it easier to turn proximal head 2219 in order to either retain or release the control wire. Distal collet assembly 2223 has a thru hole 2225 configured to receive hypotube. When hypotube is inserted into thru hole 2225, rotating head 2223 about threads 2227 in a first direction causes collet 2223 to clamp down on hypotube in a vise like grip, and rotating head 2219 in an opposite direction causes collet 2223 to release hypotube. When control wire is secured by collet 2223, collet assembly 2200 may be manipulated to exert control over the hypotube. For example, twisting collet holder control assembly 2200 may place torque on the hypotube.

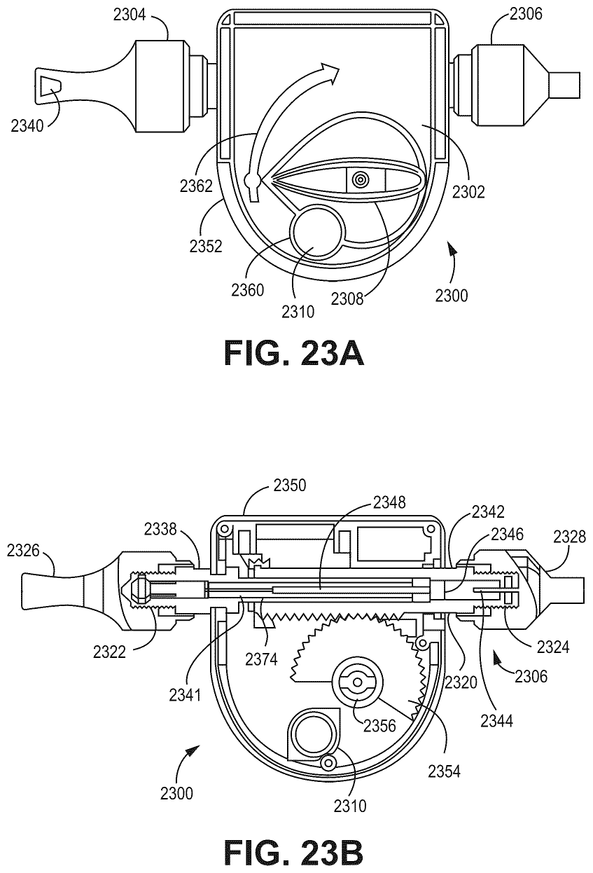

Referring to FIGS. 23A and 23B, an alternative embodiment of an actuation tool 2300 comprises a support structure 2302, a corewire connection 2304 and a hypotube connection 2306 each connected to opposite ends of the support structure 2302, a dial 2308, and a button lock 2310. Corewire connection 2304 and hypotube connection 2306 interface with support structure 2302 along a channel 2320 that provides for passage of the corewire, as shown in FIG. 23B. Corewire connection 2304 and tube connection 2306 are gripping devices that respectively grip the corewire and overtube when engaged to provide for their relative longitudinal movement through rotation of dial 2308.

Corewire connection 2304 and hypotube connection 2306 are collets that comprise, respectively, threaded receiving sleeves 2322, 2324 and mated threaded caps 2326, 2328. Receiving sleeves have a taper and one or more slits such that channels through the receiving sleeves shrink in diameter when the mated cap is tightened such that the respective collets grip the overtube or corewire upon tightening. As shown in FIG. 23B, sleeve 2322 is integral with arm 2338 that is securely attached to housing 2302. Cap 2326 can comprise a window 2340, as shown in FIG. 23A, for observing the corewire, such that it can be quickly determined if the corewire is properly loaded in the actuation tool. Referring to FIG. 23B, corewire tubular channel 2341 is connected to sleeve 2232 to form a continuous corewire path into cap 2326. Sleeve 2234 is connected to sliding arm 2342. Sliding arm 2342 has a hypotube channel 2344, a hypotube stop 2346 that provides a limit on the insertion of the hypotube with a corewire tubular channel 2348 connected to sliding arm 2342 extending beyond the hypotube stop. Corewire tubular channel 2348 has an inner diameter slightly larger than the outer diameter of corewire tubular channel 2341 so that corewire tubular channel 2348 slides over corewire tubular channel 2341 when sliding arm 2342 moves such that the corewire is supported essentially along its entire length within actuation tool 2300.

While the embodiment shown in FIG. 23 is based on collets, corewire connection 2304 and hypotube connection 2306 can be based on other designs. For example, connections 2304, 2306 can comprise clamps that snap between locked and unlocked configurations, in contrast with the collets that screw into position. In some embodiments, a lever arm can be used to transition the connections between locked and unlocked positions. Various clamps designs in the art can be adapted as substitutes for the collets based on the disclosure herein.

The components internal to the support structure 2302 comprises a control element that moves the corewire connection and the tube connection away from and toward each other to move the corewire and hypotube away from and toward each other, respectively. Support structure 2302 comprises housing 2350 and cover 2352 that attached to housing 2350 to cover the moving parts within housing 2350. The control element can include a transmission comprising gear 2354 that interfaces with sliding arm 2342 such that rotation of gear 2354 is converted to translation motion of sliding arm 2342 such that the position of corewire connection 2304 and hypotube connection 2306 can be adjusted. In particular, gear 2354 and sliding arm 2342 comprise teeth that cooperate with each other. Gear 2354 is operably connected to a knob 2356 that connects with dial 2308. When dial 2308 is rotated, gear 2354 rotates with the dial 2308 and the gear's teeth cooperate and move with the teeth of sliding arm 2342 to convert the rotational movement of the dial 2308 and gear 2354 to translational movement of sliding arm 2342 to move corewire connection 2304 relative to tube connection 2306. Other transmission designs for converting rotational motion of the rotatable element to a translational motion of the corewire connection or the overtube connection can replace the design shown in FIG. 23 if desired.

Cover 2352 comprises a first hole for the passage of a portion of knob 2356 to provide for connection to dial 2308 and a second hole 2360 for the passage of depressible button 2310. Cover 2352 can further comprise markings 2362 to provide instructions. Dial 2308 comprises a notch 2370 that engages with safety button 2310 at a particular rotation of dial 2308 to prevent rotation of the dial 2308 unless the safety button 2310 is depressed. Safety button 2310 can be constructed with a spring, such as a conventional spring structure or the like, or with other elastic material or appropriate construction. In some embodiments, a notch is positioned to engage safety button 2310 at a dial position corresponding with the deployed configuration of the fiber cartridge corresponding with a particular relative position of the corewire and hypotube. Actuation tool 2300 can be supplied with a removable shipping lock that interfaces with dial 2308 and cover 2352 to supply the dial at a particular orientation. The shipping lock can be kept in position until the fiber-based device is placed within the patient and the operator is ready to deploy the fiber cartridge. The shipping lock can be removed to deploy the fiber cartridge or other element within the patient. In alternative or additional embodiments, a second depressible button or the like can be used to hold dial 2308 at a delivery position to resist premature deployment of the device. Furthermore, other appropriate locking features, such a frictional catch or the like, can replace the button lock to provide fixed positions of the dial at the deployed and/or delivery positions of the dial.

Referring to FIG. 23B, a cut away exposed view of actuation tool 2300 reveals the components internal to sliding arm 2342 and arm 2338. Arm 2338 has a projecting sleeve 2374 that extends within sliding arm 2342 to facilitate the sliding motion of sliding arm 2342 while keeping the channel aligned for the corewire. The clearance between the adjustable corewire channel 2348 and the corewire can be less than or equal to about 0.003 inch. Adjustable corewire channel 2348 can provide appropriate support for the entire length of the corewire exposed from the hypotube through the locked position within the corewire connection 2304. In some embodiments, adjustable corewire channel 2348 extends to leave less than about 0.001 inch of the corewire unsupported between the position at which the corewire exits the overtube and the locked position in corewire connection 2304.

In operation, actuation tool 2300 is constructed to actuate or de-actuate the fiber cartridge by taking an advantage of the configuration of the integrated guiding structure with the proximal end of the corewire extending from the proximal end of the tube. The proximal end of the corewire is inserted through hypotube connection 2306 and adjustable corewire channel 2348 into the corewire connection 2304. The hypotube contacts stop 2346 to indicate full insertion of the hypotube into hypotube connection 2306. The user can observe the corewire within observation window 2340 to confirm that the corewire is properly positioned within the corewire connection 204. After the corewire is properly positioned corewire connection can be locked onto the corewire, and hypotube connection can be similarly locked onto the hypotube either before or after locking the corewire connection. A safety lock can be removed to rotate dial 2308 to deploy the fiber cartridge or other device, and dial 2308 is rotated until button 2310 extends outward to engage a notch to lock the device in the deployed configuration. When dial 2308 is rotated clockwise, gear 2354 also rotates clockwise and causes translational movement of sliding arm 2342 away from corewire connection 204, which increases the length of adjustable channel 2348 which brings the support structures of the fiber cartridge toward each other to flare the fibers. When dial 2308 is rotated counterclockwise upon depressing button 2310, gear 2354 also rotates counterclockwise and causes translational movement of sliding arm 2342 toward corewire connection 2304, which decreases the length of adjustable channel 2348 to transition the fiber cartridge to a lower profile recovery configuration. The actuation tool can be temporarily removed for the loading of other instruments over the integrated guide structure.

In addition to providing its primary function of embolic protection, the integrated guide device with an integral filter can provide additional procedural advantages. While the device is designed to have an acceptably small diameter, the diameter is still larger than a standard guidewire at the filter cartridge, which can provide some advantages for delivery of the aspiration catheter as well as performance of the aspiration. Specifically, the device can be used as a navigation aide in the following ways: reduce the ledge effect, active curvature of the wire, and an anchor. Reducing the ledge of a larger catheter at a distal opening is useful as it decreases the chance of the catheter catching on an arterial bifurcation. A common ledge effect is found in the neurovasculature at the origin of the ophthalmic artery or cerebral perforators. Two options to accomplish this is to introduce the DAISe tip into the larger catheter in a U fashion so that when the tip is partially pushed out of the catheter it forms an atraumatic U as a bumper. The U configuration could be aided with a shaped curve in the distal tip. The tip could be shaped during manufacturing or at the point of care. In some embodiments, the laser cut hypotube provides for curving the laser cut hypotube from manipulations at the proximal end. Use of the curved laser cut hypotube is described generally above and in certain embodiments further below.

Pre-shaped or shapeable tip 118 may be between 0.05 and 8.0 cm in length. In some embodiments, the tip may be pre-shaped with an angle of greater than zero to less than 360 degree range. In some embodiments, the tip may have a curving range between about 5 and about 350 degrees and in further embodiments from about 90 degrees to about 300 degrees. In alternative embodiments, the filter may be partially or fully delivered such that at least part of the filter extends past the tip of the catheter. The filter can also be used to actively navigate other devices. As described above, if the filter device is curved by over extending the corewire and eventually form a U either in the catheter or outside the catheter. The curvature may be used to aide navigation at arterial perforators or bifurcations. The device may also be torqued to select a particular vessel. Additionally, the device may be used as an anchor to aide in navigation. This could be done for portions of the procedures discussed in further detail below or until the whole system is desired to be withdrawn at the end of the procedure. The filter may be deployed distal to a larger catheter of another device with a lumen and used as a guide.

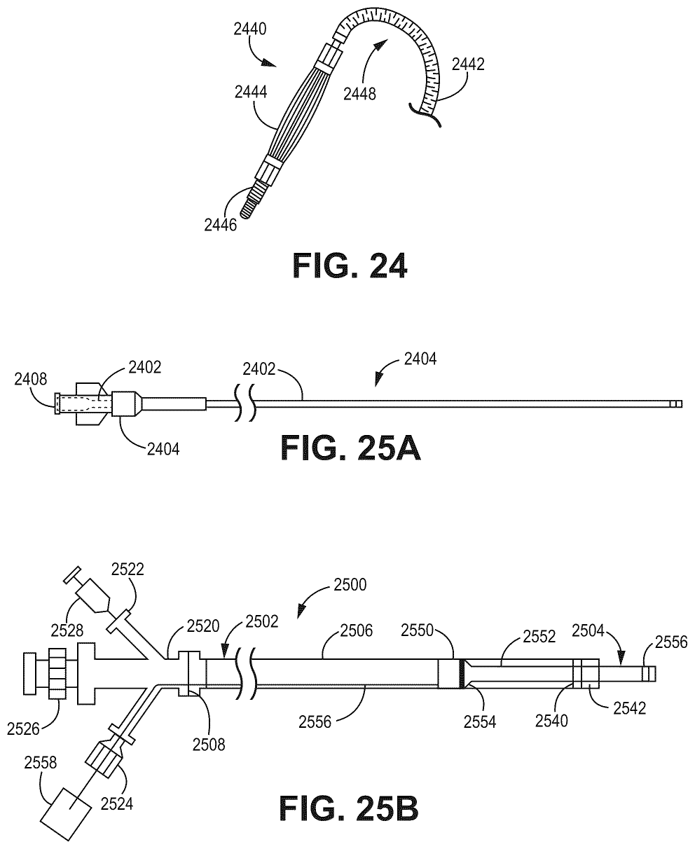

Referring to FIG. 24, an integrated embolic protection device 2440 is shown with a laser cut hypotube 2442 attached to a fiber cartridge 2444 in an undeployed configuration with a distal coil 2446 attached at a distal end of the fiber cartridge 2444. In the specific configuration illustrated, the corewire (not shown) is extended in a distal direction relative to hypotube. The tension between the hypotube and corewire results in a curving of the laser cut hypotube rather than simply stretching, as the outside of the curved hypotube stretches a greater amount than the inner curved section of the hypotube. Accordingly, laser cut hypotube 2442 has a curvature 2448 associated with the extent that the corewire is overextended, i.e., extended past the undeployed position for the filter. An advantage of the active curvature embodiments, is that the curvature can be removed by proximal movement of the corewire to return the filter to its relaxed undeployed state, which can be a desired configuration for delivery of the filter past the clot. Alternatively, the filter device may be delivered past a portion of a clot and partially or fully deployed such that the wire may be used as a guide to aide in navigating devices. Alternatively, the filter may be deployed distal to the clot and multiple passes or devices could be navigated until the vasculature procedure is near complete or the territory being treated is finished. The filter device may be withdrawn deployed and while under aspiration or deactuated and withdrawn or any possible combinations of previous.

The devices are generally assembled under sterile conditions using medical grade materials. Generally the devices can be sterilized by any suitable technique that does not damage any of the materials in the assembled device. Depending on the sterilization technique used, the device may be sterilized before or after packaging. Usually, the device would be packaged in a well-labelled easy open sterile packaging, such as those known in the art. The device may or may not be sold in different sizes designed for particular vessel size ranges, although a single device design may be suitable of expected vessel size ranges so that a single device design can be sufficient.

### Ancillary Medical Devices

As suggested above, the embolism protection devices described herein are generally used in conjunction with a medical procedure involving additional treatment devices. In particular, the present devices are useful in the context of aspiration thrombectomy, especially in the cerebral vasculature. Suitable aspiration catheters are described in the following. Other useful ancillary devices include, for example, stent retrievers and the like, which are described further below. Also, the embolism protection devices are generally delivered from a microcatheter that guides the placement of filter cartridge generally past an occlusion. As described below, guidewires can be used in parts of the procedure.

Various components for treatment of vascular occlusions can be used individually or in various groupings. Desirable groupings are described in the following along with methods of using the device and systems, especially for acute ischemic stroke treatments. The procedures generally make use of an aspiration catheter that is positioned proximal to the occlusion such that aspiration can be applied during selected portions of the procedure, such as steps of the procedure that can generate emboli. A filter device can be placed distal to the occlusion and can catch at least some or all relevant emboli if they flow downstream as well as possibly contributing to the manual removal of thrombus. In some embodiments, the filter device and aspiration catheter can set boundaries on the treatment zone, and additional components may or may not be used, such as stents and/or stent retrievers, within the treatment zone. In some embodiments, liquid can be profused into the treatment zone to make up for at least some fluid removed from the treatment zone by aspiration or to provide hydraulic forces to assist with the clot removal.

The basic systems for the procedures described in this section comprise a suction catheter and an embolic protection device mounted on an integrated guide structure, as described in detail above. Other basic devices can include, for example, a guide wire, a guide catheter and a microcatheter. Use of these devices are described as a system for some examples of procedures. In a further set of example procedures, stents and stent retrievers can also be used for the procedures.

Guidewires for neurovascular applications are commercially available. These include TRANSEND® (Stryker) with a distal outer diameter (OD) of 0.014 inches (0.36 mm) and 0.0155 in (0.40 mm) proximal, SYNCHRO® (Stryker) with a range of diameters, CHIKAI™ (Asahi Intecc) with a 0.36 mm diameter and HEADLINER® (MicroVention/Turumo) with a range of diameters available. A guidewire for cerebral vessels with a hyperbolic corewire grind is described in U.S. Pat. No. 10,518,066 to Pokorney et al. (hereinafter the '066 patent), entitled “Medical Guidewires for Tortuous Vessels,” incorporated herein by reference.

The '066 patent describes not only desirable guidewire embodiments for cerebral vessels, but also an extendable guidewire that can provide for effective delivery of the tip of the guidewire deeper into the vasculature. As described above, improvements in this guidewire design provide for improved flexibility while balancing continued pushability along with the capability of active curving. This improved guidewire can improve reach of the guidewire into smaller vessels. Microcatheters have been designed to allow for access to small blood vessels, such as cerebral blood vessels. Various commercially available microcatheters are available for use in the neurovasculature including, for example, Marathon™ (Covidien/Medtronic, 1.3Fr distal OD), Echelan™ (Covidien/Medtronic, 1.7Fr distal OD), Nautica™ (Covidien/Medtronic, 2.2Fr distal OD), Sofia® (Microvention/Turumo), Spinnaker Elite™ (Boston Scientific Co.), and Excelsior® (Stryker, 1.7Fr distal OD). These microcatheters or similar devices can be used for the procedures herein, and for appropriate embodiments, these structures can be adapted for use for stent and/or stent retriever delivery. Of course the term microcatheter can cover a range of devices, and the present discussion can focus on catheters useful for the procedures described herein. In some embodiments, microcatheters can comprise a distal section that is narrower than a proximal section. However, in further embodiments, a microcatheter can have an approximately constant diameter along its length to facilitate delivery of other devices over the microcatheter. A narrow distal diameter allows for the catheter to navigate the tortuous vessels of the brain. The distal section can be highly flexible enough to navigate the vessels, but resilient enough to resist kinking. A microcatheter comprises at least one lumen. The microcatheter can then be used to deliver other treatment devices, aspiration, therapeutic agents, or other means of treating a condition. While microcatheters can have a selected size, in some embodiments, the microcatheters can have a distal outer diameter from about 1.0Fr to about 3.5Fr and in further embodiments from about 1.5Fr to about 3Fr, and a length from about 30 cm to about 200 cm and in further embodiments from about 45 cm to about 150 cm. A person of ordinary skill in the art will recognize that additional size ranges within the explicit ranges above are contemplated and are within the present disclosure.

Microcatheters can be formed, for example, with a polymer tube generally with at least a portion of which having metal reinforcement. A representative microcatheter is shown in FIG. 25A. Microcatheter 2400 comprises a tubular body 2402 that generally comprise polymer with optional metal reinforcement reflowed into the polymer, and hub 2404. Tubular body 2402 can have a marker band 2406 that facilitates positioning the catheter in a blood vessel. Hub 2404 can have a Luer connector 2408.

An aspiration catheter can be an effective component for the removal of cerebral clots, even if used alone, especially for softer clots. When aspiration catheters are combined with the other elements described herein, the combined treatment systems can offer several elements in the cooperative efforts to remove the clot, although the aspiration catheter may be effective when used alone. The aspiration catheter provides removal forces from the proximal side of the treatment system while a filter device can provide the distal backstop, although a fiber based filter can in principle be extended in the clot to engage the clot for pulling.

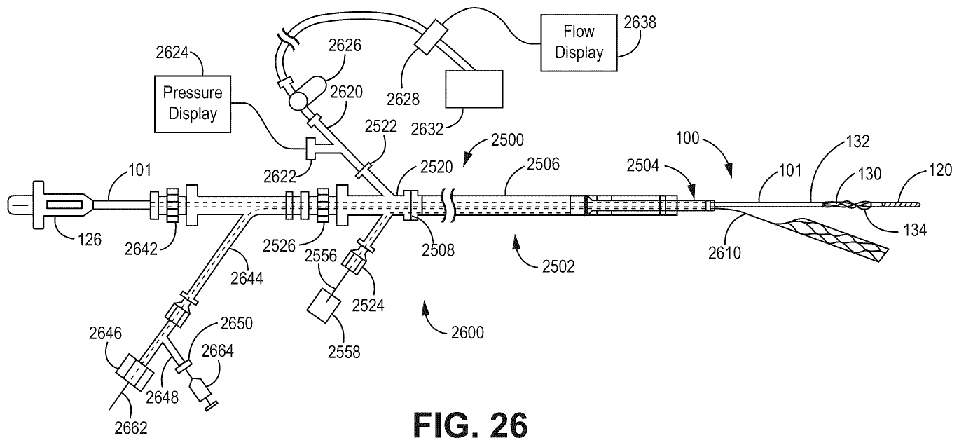

Various aspiration catheters have been developed for providing improved suction within the narrow tortuous vessels, such as vessels of the cerebral vasculature. In some embodiments, these aspiration catheters have a narrowed distal tip that can reach into narrow vessels but provide high flows out of the vessel due to the larger proximal lumen. These improved designs are described in U.S. Pat. No. 10,058,339 to Galdonik et al., entitled “Aspiration Catheters for Thrombus Removal,” incorporated herein by reference. Aspiration catheters with a narrowed distal segment approved and commercially available for neurovascular procedures include Zoom™ catheters (Imperative Care) and MAX™ catheters (Penumbra). A new design is based on the use of a guide catheter to function as a part of aspiration lumen with a narrowed extension of the aspiration catheter extending from the guide catheter. See the '915. patent cited above. An embodiment of a Q™ Catheter (MIVI Neuroscience) is shown in FIGS. 25-26.

Referring to FIG. 25B, aspiration system 2500 comprises an aspiration adapted guide catheter 2502 and an aspiration catheter 2504. Aspiration adapted guide catheter 2502 comprises tubular shaft 2506 and hub 2508. Hub 2508 generally is connected to proximal fittings 2520 that can comprise various branched manifolds and ports, which may be also suitable for use also as a handle. Proximal fittings 2520 can comprise a port 2522 for connection of an aspiration source, such as a syringe or pump, and an optional control wire port 2524, as well as possibly other additional ports and/or fittings to provide desired functionality and access, in which all such ports and fittings can be arranged in a branch configuration or other suitable configuration. Proximal fitting 2520 can comprise a suitable hemostatic valve 2526 or the like to provide for entry of a guidewire and/or structures delivered over the guidewire into the guide catheter lumen, such as alternative treatment structures and/or embolic protection devices, such as the embolic protection device described herein. Interfacing of aspiration system 2500 with additional components is described further in the context of FIG. 26. As shown in FIG. 25B, a negative pressure device 2528 is shown connected with suction port 2522, and suitable negative pressure devices include, for example, syringes, pumps, such as peristaltic pumps, piston pumps or other suitable pumps, aspirator/venturi, or the like.

Tubular shaft 2506 can have an approximately constant diameter along its length, or the guide catheter can have sections with different diameters, generally with a smaller diameter section distal to a larger diameter section. Tubular shaft 2506 can have one or more radiopaque marker bands to facilitate positioning of the tubular shaft within the patient, and FIG. 25B shows a marker band 2540 near the distal end of tubular shaft 2506, although additional positions and/or alternative positions can be used as desired. At or near the distal end of the shaft, a stop 2542, such as a ring or inner notch, can be positioned to retain a portion of aspiration catheter 2504 within the lumen of tubular shaft 2506.

Aspiration catheter 2504 can comprises an interface section 2550, distal section 2552, transition section 2554 and control structure 2556, such as a control wire. Interface section 2550 is configured to interface with guide catheter 2502 along the inner surface of tubular shaft 2506. All or a part of interface section 2550 can be configured to remain within the lumen of guide catheter 2502. Distal section 2552 is shown with radiopaque marker band 2556 near the distal tip of distal section 2552, although aspiration catheter 2504 can comprise a plurality of radiopaque marker bands at appropriate locations if desired. Control structure 2556 can be a control wire or the like that connects with interface section 2550 and extends exterior to the guide catheter, such as exiting through control wire port 2524. Control structure 2556 can be used to control positioning of interface section 2550 within the lumen of tubular shaft 2508. Control structure 2556 can comprise a control tool 2558, such as a handle, slide or the like that can anchor a control wire or other connecting element to facilitate movement of the control wire. In some embodiments, the clearance can be made sufficiently small between the outer surface of proximal portion 2540 and the inner surface of tubular shaft 2508 that a separate seal is not needed.

The guide catheter can have an outer diameter from about 5.5 Fr (1.667 mm diameter) to about 10 Fr (3.333 mm diameter), in further embodiments from about 6 Fr (1.833 mm diameter) to about 9 Fr (3 mm diameter), and in some embodiments from about 6.25 Fr (2 mm diameter) to about 8.5Fr (2.833 mm diameter). The guide catheter measurement are generally referenced to the outer diameter, and the inner diameter is less than the outer diameter by twice the wall thickness. The length of the guide catheter can be from about 30 cm to about 150 cm, in further embodiments from about 35 cm to about 130 cm and in additional embodiments from about 40 cm to about 120 cm. The length of aspiration catheter 2504 can be from about 30 cm to about 150 cm, in further embodiments from about 35 cm to about 130 cm and in additional embodiments from about 40 cm to about 120 cm. A person of ordinary skill in the art will recognize that additional ranges of dimensions within the explicit ranges above are contemplated and are within the present disclosure.

The outer diameter at the tip of the aspiration catheter generally is (diameter in mm=(Fr value)/3. Fr represents the French catheter scale) at least about 0.5Fr less than the outer diameter of the interface section of the aspiration catheter. The smaller diameter of the distal section can provide access to desirable vessels, such as cerebral vessels. It was previously discovered that good suction properties could be obtained with an aspiration catheter with a stepped down diameter in a distal section. Thus, for example, the majority of the length of the aspiration catheter can be 6Fr outer diameter while a distal section may be 5Fr outer diameter, which roughly corresponding decreases in the inner diameters. A person of ordinary skill in the art will recognize that additional ranges of dimensions within the explicit ranges above are contemplated and are within the present disclosure.

FIG. 26 depicts a view of an assembled embodiment of a treatment system with components to assist in a procedure. Specifically, treatment system 2600 comprises embolic protection device 100, aspiration system 2500, an optional stent retriever 2610 and various fittings and ancillary elements described below, in an assembled structure. The components all interface through proximal fittings 2620. A distal portion of the system is shown illustrating aspiration catheter 2504 extending from guide catheter 2502. A proximal portion of the treatment system shows proximal fittings 2520 of aspiration system 2500 extending proximally from guide catheter 2502. Additional features and various design options are described in the context of FIG. 25B above, and these can be adapted appropriately for use in treatment system 2600.

As depicted in FIG. 26, the distal portion of embolic protection device 100 extends out from a distal opening of aspiration catheter 2504. In some embodiments for use, filter cartridge 130 is deployed past a clot, while aspiration catheter is deployed with its distal opening proximal to the clot. At the proximal end of the structure, hypotube 101 is seen extending past proximal fittings 2620 and connecting to actuator 126 with the corewire supported to avoid kinking. While a particular embodiment is shown for convenience, any of the embolic protection devices described above can be similarly incorporated into treatment system 2600.

The depiction of stent retriever 2610 in FIG. 26 suggests the use generally of a suitable atherectomy device in conjunction with aspiration catheter 2504 and embolic protection device 100 in therapy system 2600. Desirable thrombus engagement devices are described in the '386 patent cited above. Stent retrievers are commercially available for acute ischemic stroke treatment. For example, stent retrievers are available from Medtronic Corp. (Solitaire®,) and Stryker (TrevoProvue™). Stent retrievers are described, for example, in U.S. Pat. No. 8,795,305 to Martin et al., entitled “Retrieval systems and methods of use thereof,” incorporated herein by reference.

In comparison with FIG. 25B, treatment system 2600 in FIG. 26 includes more features associated with the proximal fittings to provide desired functionality. In embodiments, proximal fittings 2620 have various branches providing desired functionalities as described in the several embodiments presented herein. In embodiments, a first branched manifold corresponds with proximal fittings 2520 and is connected at the distal end of guide catheter 2502, which is shown as a three-branched manifold, although it can be provided as two Y-branch manifolds each with two branches connected in series. In treatment system 2600, port 2522 is connected to a more elaborate aspiration control setup. A y-branch manifold 2620 connected to port 2522 comprises a pressure sensor 2622 on one branch, and pressure sensor 2622 is connected to a pressure display 2624. The other branch of y-branch manifold 2620 connects to an inline filter 2626 configured to remove any thrombus within the line to the negative pressure source, flow meter 2628 connected to a flow display 2630, and a negative pressure source 2632. Suitable negative pressure sources include, for example, syringes, pumps, such as peristaltic pumps, piston pumps or other suitable pumps, aspirator/venturi, or the like, and commercial pumps are available for this purpose, such as Gomco Aspiration Pumps available from MIVI Neuroscience.

As depicted in FIG. 26, a further y-branch manifold 2640 is connected to hemostatic valve 2526, although hemostatic valve 2526 can be replaced by a Luer connector is y-branch manifold 2640 has a suitable mated connector. The various manifolds depicted can be reconfigured in various ways using components known in the art to provide comparable functionality. In the depiction of FIG. 26, y-branch manifold 2640 terminates with a hemostatic valve 2642 and has a branch 2644 ending in a hemostatic valve 2646 and a branch 2648 with a connector 2650. A control wire 2662 for controlling stent retriever 2610 exits hemostatic valve 2646. An infusion syringe 2664 is connected to connector 2650, and syringe 2664 can be filled with contrast dye, medication or other appropriate biologically compatible fluid for delivery into the patient.

Aspiration catheters with control features based on pressure sensors and/or flow sensors are described in copending U.S. patent application Ser. No. 17/667,828 to Wainwright et al. (hereinafter the '828 application), entitled “Suction Catheter Systems With Designs Allowing Improved Aspiration and Evaluation of Aspiration Condition,” incorporated herein by reference.

### Use of Embolic Protection Device-Aspiration Atherectomy

In general, the embolic protection device described herein can be used in any procedure where the risk of emboli warrants it. While the device is designed for use in more challenging vessels, the embolism protection device can also be used in less challenging delivery contexts. However, the device can be particularly useful in performance of aspiration atherectomy, especially for the treatment of acute ischemic stroke events. The following discussion focuses on such procedures, and the use of the device in other contexts can be adapted from this discussion.

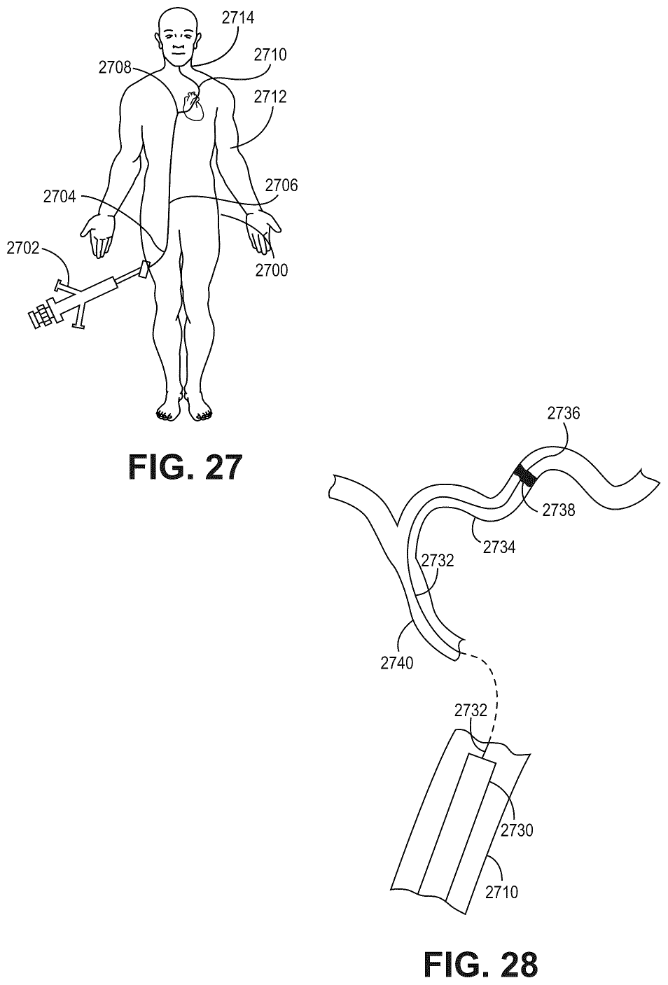

Referring to FIG. 27, a human patient 2700 is shown with a treatment system 2702 inserted into their femoral artery 2704 where it is guided up the descending aorta 2706 to the ascending aorta 2708 where it is guided into a carotid artery 2710 (left or right) prior to reaching the heart. The distal end of the system is then guided through the patient's neck into an internal carotid artery and then into the cerebral arteries forming the neurovasculature. While this can be a desirable approach to the cerebral arteries, alternative access locations include the arm 2712 or the neck 2714. While human patients are of particular interest, the devices can be used for farm animals or pet animals.

In the basic procedures described herein, generally an aspiration catheter and a fiber-based embolic protection device are used. Procedures can be grouped into two classes, with a first class involving use of a microcatheter and a second class involving direct delivery of the integrated guide structure. Selection of the particular procedure may depend on the particular vessel, characterization of the clot, preference of the provider, regulatory issues or the like.

In the basic procedure involving an aspiration catheter and a fiber based embolic protection device delivered through a microcatheter, the basic steps are:
1. Place guide catheter in place.
2. Extend guidewire to treatment site with tip past the occlusion.
3. Place aspiration catheter in position.
4. Guide microcatheter into position with distal tip past occlusion.
5. Remove guidewire.
6. Insert fiber-based filter element on an integrated guide structure through microcatheter.
7. Push fiber-based filter out of the microcatheter, deploy the filter past occlusion and confirm deployment with imaging. The microcatheter may be removed after placement of the filter cartridge.
8. Apply aspiration for clot removal. Aspiration profile can be adjusted to improve clot removal efficacy.
9. To further facilitate clot removal, the aspiration catheter can be moved toward a fixed deployed filter, the fiber-based filter can be pulled toward a stationary aspiration catheter, or both the aspiration catheter and the fiber-based filter can be moved towards each other.
10. Depending on the status of the clot, the fiber-based filter can be partially or completed collapsed into a lower profile configuration for removal.
11. Remove devices from patient. A collapsed filter may or may not be brought into the lumen of the aspiration catheter for removal form the patient. It can be desirable to remove the filter under aspiration without de-actuating the filter to collapse it. In some embodiments, the filter and aspiration catheter may be removed from the patient while maintaining a fixed distance between the filter and the aspiration catheter while the filter and aspiration catheter are withdrawn. The filter may be brought into the lumen of the guide catheter for completion of removal or removed adjacent the guide catheter as both are removed. Aspiration may be maintained until the devices are clear of the patient.

In a basic procedure involving delivery of the filter structure without a microcatheter, the basic steps are:
1. Place guide catheter in place.
2. Deliver to the end of the guide catheter, an aspiration catheter with the integrated guide structure having the filter positioned in the aspiration catheter.
3. Advance the aspiration catheter and integrated guide structure together past the guide catheter. Generally, the integrated guide structure is positions to reduce or eliminate a ledge effect caused by the catheter.
4. Position the distal end of the aspiration catheter at a target position proximal to the clot.
5. Continue advancing the integrated guide structure past the aspiration catheter to advance the filter cartridge past the clot.
6. Deploy the filter such that the fibers extend to the vessel wall and confirm deployment with imaging.
7. Apply aspiration for clot removal. Aspiration profile can be adjusted to improve clot removal efficacy.
9. To further facilitate clot removal, the aspiration catheter can be moved toward a fixed deployed filter, the fiber-based filter can be pulled toward a stationary aspiration catheter, or both the aspiration catheter and the fiber-based filter can be moved towards each other.
10. Depending on the status of the clot, the fiber-based filter can be partially or completed collapsed into a lower profile configuration for removal.
11. Remove devices from patient. A collapsed filter may or may not be brought into the lumen of the aspiration catheter for removal form the patient. It can be desirable to remove the filter under aspiration without de-actuating the filter to collapse it. In some embodiments, the filter and aspiration catheter may be removed from the patient while maintaining a fixed distance between the filter and the aspiration catheter while the filter and aspiration catheter are withdrawn. The filter may be brought into the lumen of the guide catheter for completion of removal or removed adjacent the guide catheter as both are removed. Aspiration may be maintained until the devices are clear of the patient.

While this order of steps accounts for practical implementation and provides an overview of the procedure, the precise order is not sacrosanct, as will be recognized by a person of ordinary skill in the art. Thus, appropriate steps may be performed in a different order, and some steps can be performed in substeps that may be interspersed with portions of other steps. For example, positioning of the aspiration catheter may be partially performed prior to placement of the guidewire, while the further positioning of the aspiration catheter may be performed later in the process.

In some embodiments, contrast may be injected through the microcatheter at one or more stages of the procedure to check flow in the vicinity of the procedure. The aspiration catheter may be advanced adjacent a proximal end of the occlusion and aspiration applied. For example, aspiration may be applied for about 60 to 120 seconds. If there is no flow in the aspiration tubing. the aspiration catheter can be slowly pulled in the proximal direction while leaving the filter in place. If flow returns, the aspiration catheter may be re-advanced towards the occlusion. If flow does not return, the aspiration catheter may be fully withdrawn, while leaving the filter in place, flushed, reinserted, and readvanced to the proximal face of the occlusion. Aspiration may again be applied, for example, for 60 to 120 seconds. By repeating these steps, an occlusion may be removed by aspirating smaller pieces of the occlusion one at a time. In this usage, the filter acts as a tether anchoring the wire, permitting the aspiration catheter to be easily withdrawn and readvanced to the occlusion. In instances where it is difficult to advance the aspiration catheter, the microcatheter may be advanced over the wire reduce the ledge effect or a smaller aspiration catheter may be used. Repositioning of various components can take place through the procedure as appropriate and desired by the user.

Instrumentation of the aspiration flow can be used to guide the aspiration process. In particular, flow and or pressure can be measured to help evaluate process steps. See the '828 application cited above.

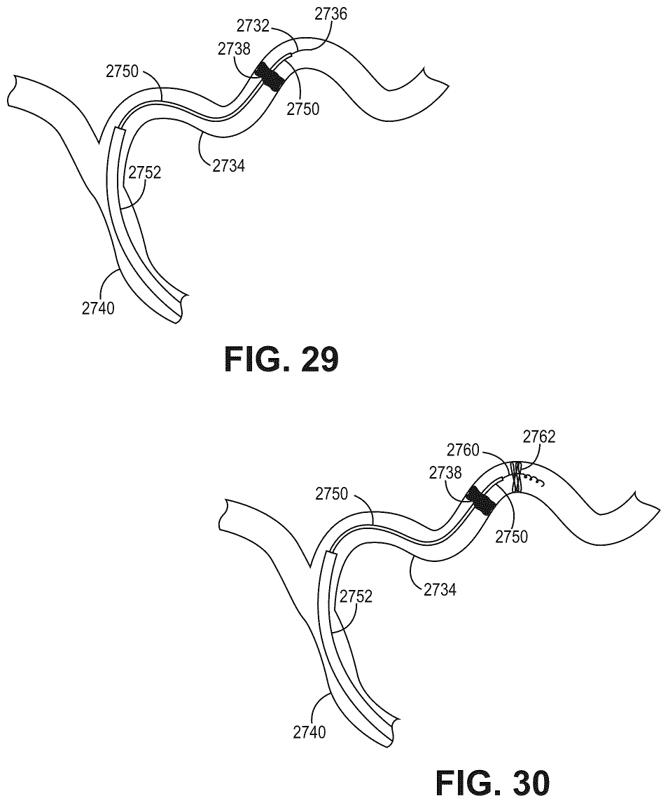

Referring to FIG. 28, guide catheter 2730 is placed in the carotid artery 2710. A guidewire 2732 is guided past carotid artery 2710 into a cerebral artery 2734 with its distal tip 2736 positioned past a clot 2738. A vascular path truncated in the figure from carotid artery 2710 (generally an internal carotid artery) to an upstream cerebral artery 2740 branching into cerebral artery 2734 is depicted with a dashed line to note portions of the path not depicted for simplicity of drawing. Referring to FIG. 29, a microcatheter 2750 is positioned over guidewire 2732 with its distal tip past clot 2738. Aspiration catheter distal tip 2752 is positioned entering cerebral vessel 2734. Depending on the specifics of the vasculature and the aspiration catheter design, aspiration catheter distal tip may be brought closer or further from clot 2738. The medical professional can adjust the procedure accordingly based on the position of the aspiration catheter.

With microcatheter 2750 in position, guidewire 2732 can be removed and an integrated guide structure 2760 with a fiber based filter element 2762 can be put in position with fiber based filter element 2762 deployed distal to clot 2738. Referring to FIG. 30, fiber based filter element 2762 is shown in a deployed position past clot 2738. In embodiments in which other treatment components are not used for clot removal, aspiration can be initiated through aspiration catheter distal tip 2752, and fiber based filter element 2762 provides a back stop to catch any significant emboli that may break off from the clot during removal. For harder clots in particular, additional efforts to free or fragment the clot, such as pulsatile aspiration and/or mechanically engaging the clot may generate a risk of emboli generation.

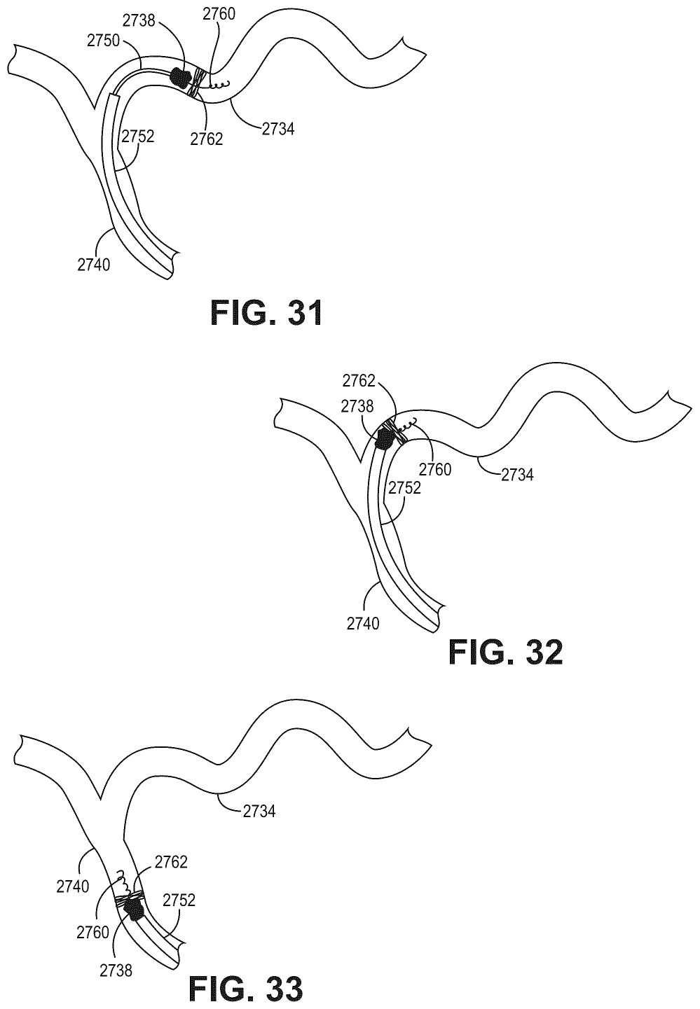

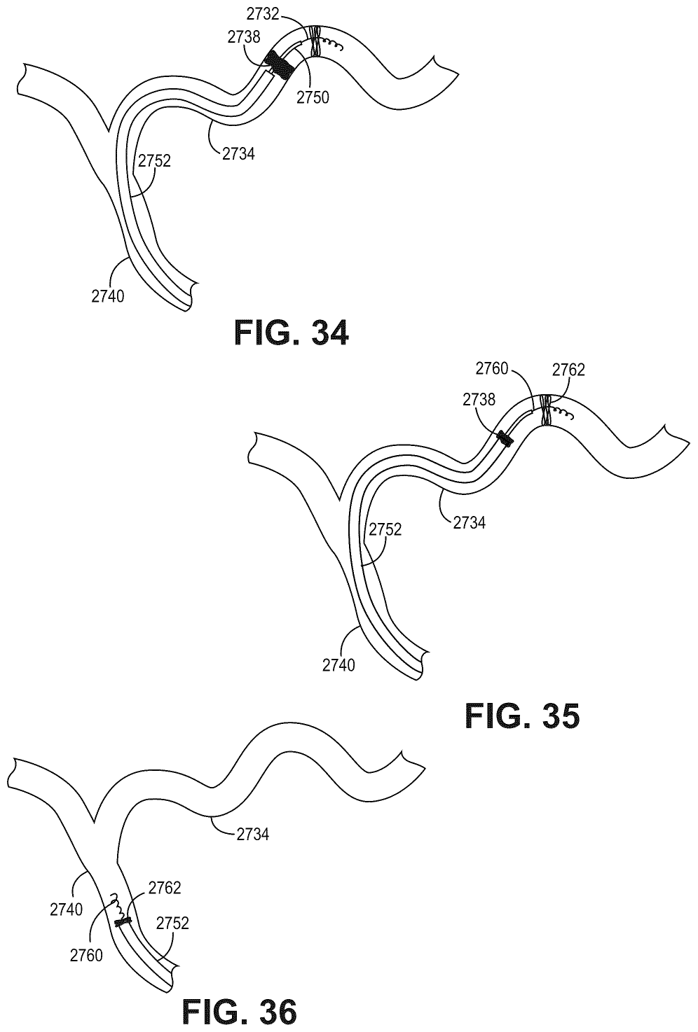

With the filter as a backstop protecting against emboli generation, the aspiration catheter and embolic protection device can be manipulated to increase the efficacy of the clot remove, for example, by either moving the deployed filter toward the aspiration catheter or bringing the aspiration catheter up to the clot. The procedure of pulling the deployed filter toward the aspiration catheter is depicted in FIGS. 31-33. The procedure of advancing the aspiration catheter to the clot is depicted in FIGS. 34-36. Any reasonable combination of these procedures can be similarly used.

Referring to FIGS. 31-33, fiber based filter element 2762 in a deployed configuration can be pulled in a proximal direction toward the aspiration catheter distal tip 2752. Engaged clot 2738 may be pulled by fiber based filter element 2762, or loosened clot 2738 can be more directly carried to the aspiration catheter by the aspiration. Clot 2738 upon engagement may generate fragments that may embolize. Any fragments may be removed by the aspiration, or they may be trapped by fiber based filter element 2762 for subsequent removal. While FIG. 43 shows fiber based filter being deployed distal to the clot, the fiber based filter can be deployed at the clot so that the fiber directly engages the center of the clot.

Referring to FIG. 34, aspiration catheter distal tip 2752 is brought up the clot 2738 while fiber based filter element 2762 remains stationary. Referring to FIG. 35, aspiration with the aspiration catheter distal tip 2752 adjacent the tip is depicted as being effective to draw cot 2738 into the catheter. With the clot sufficiently removed though aspiration catheter 2752, fiber based filter element 2762 can be transitioned to a lower profile configuration by advancing the corewire in a distal direction relative to the hypotube and drawn fully or partially into aspiration catheter 2752, as shown in FIG. 36. Then, treatment system 2702 can be removed from the patient. In some embodiments, aspiration can be applied during the removal of the filter.

The amount of fluid aspirated can be selected by the medical professional to achieve desired performance. A moderate amount of blood may be removed, and with the suction available through commercial aspiration catheters, some trauma to the blood vessel may result from the pressure changes associated with the aspiration and the termination of the aspiration. To ameliorate these pressure changes at least to some degree, fluid can be perfused through the microcatheter. The fluid can be buffered saline, blood (either from the patient or a compatible donor) or other suitable biocompatible fluid. Due to the smaller diameter of the microcatheter, the perfused fluid may not compensate for the aspirated fluid, but reduction of pressure changes can be desirable. Also, the perfused fluid can further flush any clot fragments toward the aspiration catheter to lower risk of distal embolization.

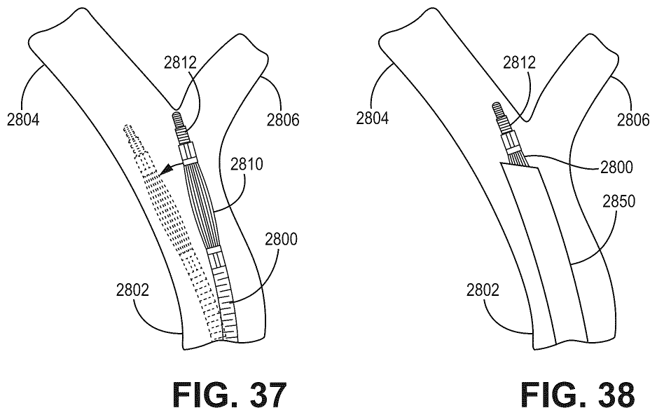

As noted above, the embolic protection device with the distal laser cut hypotube section is susceptible to curvature upon extension of the corewire distally relative to the hypotube. This curvature can be used to facilitate device delivery. An example application is shown in FIG. 37. Embolic protection device 2800 is depicted in vessel 2802 that flows into primary branched vessel 2804 and secondary ranched vessel 2806. In an attempt to direct filter cartridge 2810 into primary branched vessel 2804, a ledge effect can be encountered in which tip 2812 extending from filter cartridge 2810 contacts the branch at secondary branched vessel 2806. Curving embolic protection device 2800 by advancing the corewire can pivot tip 2812 to avoid the ledge effect, as shown in FIG. 37, in which curvature pivots tip 2812 in a right to left transition.

Referring to FIG. 38, embolic protection device 2800 is delivered in conjunction with aspiration catheter 2850. As shown in FIG. 38, curvature of embolic protection device 2800 provides an angled orientation of tip 2812 and filter cartridge 2810 at the distal opening of aspiration catheter 2850. This orientation of tip 2812 and filter cartridge 2810 reduces the chance of encountering a ledge effect during delivery of embolic protection device 2800 and aspiration catheter 2850 together into the vessel.

### Further Inventive Concepts

A1. An embolic protection device with a flexible fiber-based filter element comprising:
   a corewire having a proximal section with a first diameter, a distal section having a second diameter, and a tip section having at least a segment having a non-circular cross section;
   a hypotube with a proximal section free of laser cuts and a distal section having laser cuts through the wall of the hypotube, wherein the corewire extends through the hypotube with the proximal end and the distal end of the corewire extending from respective ends of the hypotube; a stake having a central lumen with a non-circular cross section along the inner diameter, wherein the non-circular cross section of the inner diameter is shaped to engage the non-circular cross section of the tip section of the corewire, wherein the stake is interfaced with the corewire to prevent relative rotation of the stake and the corewire without limiting sliding of the corewire relative to the stake, and wherein the stake is secured with the hypotube to resist relative rotation of the stake and the hypotube;
   a fiber bundle comprising a bundle of fibers each having a first end and a second end, a first attachment element, and a second attachment element, wherein the first attachment element secures the first end of the fibers and the second attachment element secures the second end of the fibers, wherein the first attachment element is secured to the distal section of the hypotube, wherein the fiber bundle has an initial undeployed configuration with the fibers aligned and a deployed configuration with the fibers bent and the corewire in a proximal position relative to the hypotube in the undeployed configuration; and
   a distal tip secured with the second attachment element and/or to the corewire.
A2. The embolic protection device of inventive concept A1 wherein the stake is within the hypotube along the laser cut section, has an asymmetric outer surface with non-circular cross sections, and is secured in place by polymer extending from the exterior of the laser cut hypotube through the cut openings.
A3. The embolic protection device of inventive concept A2 comprising reflowed thermoplasic elastomer over at least a portion of the laser cut hypotube and wherein the reflowed thermoplastic elastomer extends through cut openings of the hypotube to engage the non-circular outer surface of the stake to provide the rotation resistance of the hypotube relative to the stake.
A4. The embolic protection device of inventive concept A2 wherein the first attachment element comprises a laser cut marker band.
A5. The embolic protection device of inventive concept A4 wherein the laser cut marker band is attached at its proximal end to the laser cut hypotube and the fibers are attached over the exterior of the laser cut marker band at an attachment section, wherein the fibers are secured with adhesive and polymer shrink wrap.
A6. The embolic protection system of inventive concept A4 wherein the second attachment element also comprises a laser cut marker band and wherein the distal tip comprises a distal coil and a blunt end.
A7. The embolic protection device of inventive concept A1 wherein the stake is also a marker band for the fiber cartridge, and wherein the marker band comprises a crimp section at which an inner lumen of the marker band has a non-cylindrical cross section to engage a corresponding portion of the corewire to provide the torque coupling.
A8. The embolic protection device of inventive concept A7 wherein the marker band has a collar for attachment to the laser cut hypotube, a fiber attachment section, wherein the crimp section is between the collar and the fiber attachment section, and a central section distal to the fiber attachment section positioned under but not attached to the fibers oriented toward the center of the filter cartridge.
A9. The embolic protection device of inventive concept A1 wherein the fibers are attached at the first attachment structure and the second attachment structure on marker bands with adhesive and shrink wrap to secure the fibers.
A10. The embolic protection device of inventive concept A1 wherein the first attachment structure and the second attachment structure comprise marker bands, wherein each marker band comprises a central section within the fiber bundle but not attached to the fibers such that the central sections meet in a deployed filter configuration.
A11. The embolic protection device of inventive concept A1 wherein 90% of the last 5 cm of the hypotube are laser cut.
A12. The embolic protection device of inventive concept A1 wherein the fiber bundle comprises at least about 100 surface capillary fibers distributed around the circumference of the corewire with a twist of the distal end relative to the proximal end of at least about 90 degrees and wherein the distal tip comprises a distal coil and a blunt tip.
A13. The embolic protection device of inventive concept A1 wherein the fiber bundle comprises radiopaque metal elements.
A14. The embolic protection device of inventive concept A1 wherein the laser cut hypotube has a spiral intermittent cut.
A15. The embolic protection device of inventive concept A14 wherein the fraction of metal cut at a section near the distal end is greater than a section at a proximal end of the laser cut hypotube to introduce greater flexibility at the distal end.
B1. An embolic protection device with a flexible fiber-based filter element comprising,
   a corewire having a distal end and a proximal end;
   a hypotube comprising a metal shaft with a lumen, wherein the corewire extends through the lumen of the hypotube with the proximal end and the distal end of the corewire extending from respective ends of the hypotube;
   a fiber bundle comprising a bundle of fibers each having a first end and a second end, a first attachment element, and a second attachment element, wherein the first attachment element secures the first end of the fibers and the second attachment element secures the second end of the fibers, wherein the first attachment element is secured to the hypotube, wherein relative movement of the corewire and the hypotube can transition the filter cartridge from a low profile delivery confirmation to a deployer filtering conformation, wherein the fiber bundle has an initial undeployed configuration with the fibers aligned and a deployed configuration with the fibers bent and the corewire in a proximal position relative to the hypotube in the undeployed configuration; and
   a distal tip comprising a distal coil secured with the second attachment element and/or to the corewire,
   wherein the first attachment element comprises a first marker band having laser cut walls over at least a portion of its length, wherein the first marker band is formed from a highly radiopaque metal.
B2. The embolic protection device of inventive concept B1 wherein the fibers comprise polymer fibers and radiopaque metal fibers.
B3. The embolic protection device of inventive concept B1 wherein the first attachment and second attachments each comprise a potted portion where the fibers are held in place with an adhesive and a shrink wrapped portion where the fibers are held in place with shrink wrap.
B4. The embolic protection device of claim B wherein the second attachment element comprises a second marker band formed from a highly radiopaque material.
B5. The embolic protection device of inventive concept B4 wherein the first and second marker bands have laser cut walls.
B6. The embolic protection device of inventive concept B4 wherein the second marker band comprises a plurality of fenestrations at an end portion, an attachment portion proximal to the end portion at which the fibers are secured, and a central portion proximal to the attachment portion within the fiber bundle but not attached to the fibers oriented to the center of the fiber bundle.
B7. The embolic protection device of inventive concept B1 wherein the first marker band comprises a collar, wherein the collar is secured to the hypotube.
C1. An embolic protection device with a flexible fiber-based filter element comprising:
   a corewire having a proximal section with a first diameter, a distal section having a second diameter less than the first diameter, and a tip section having a non-circular cross section over at least a portion of its length;
   a hypotube with a proximal section free of laser cuts and a distal section having laser cuts through the wall of the hypotube, wherein the corewire extends through the hypotube with the proximal end and the distal end of the corewire extending from respective ends of the hypotube; a friction reducing structure comprising a polymer associated with the hypotube or a mechanical element having a low friction channel, located between the corewire and a portion of the hypotube distal section;
   a torque coupler restricting rotation of the corewire and the hypotube at the torque coupler while allowing at least some sliding of the corewire within the hypotube;
   a fiber bundle comprising a bundle of fibers each having a first end and a second end, a first attachment element, and a second attachment element, wherein the first attachment element secures the first end of the polymer fibers and the second attachment element secures the second end of the fibers, wherein the first attachment element is secured to the distal section of the hypotube, wherein the fiber bundle has an undeployed configuration with the fibers aligned and a deployed configuration with the fibers bent and the corewire in a distal position in the hypotube in the undeployed configuration relative to the position in the deployed configuration; and
   a distal tip secured with the second attachment element and/or to the corewire,
   wherein advancing the corewire within the hypotube in a distal direction with the fiber bindle in an undeployed configuration results in curving of the hypotube along the distal section. C2. The embolic protection device of inventive concept C1 wherein the friction reducing agent comprises a coil.
C3. The embolic protection device of inventive concept C1 wherein the friction reducing agent comprises a low friction coating on the interior of the laser cut hypotube.
C4. The embolic protection device of inventive concept C1 comprising reflowed thermoplasic elastomer over at least a portion of the laser cut hypotube, wherein the reflowed thermoplastic elastomer extends through cut openings of the hypotube, and wherein the friction reducing agent comprises reflowed thermoplastic elastomer extending through the cut openings.
C5. The embolic protection device of inventive concept C1 wherein the torque coupler comprises a stake with a non-circular outer surface with the reflowed thermoplastic polymer holding the stake to provide fixation of the hypotube relative to the stake, and wherein a portion of the corewire having a non-circular cross section passes through a non-cylindrical channel of the stake to provide rotational coupling of the corewire and the hypotube at the stake.
C6. The embolic protection device of inventive concept C1 wherein the hypotube has at least 90% of the last 5 cm of its distal end being laser cut, and the last cut comprising an intermittent spiral cut.
C7.

The embolic protection device of inventive concept C1 wherein the embolic protection device comprises any of the features of claims 1 to 16 or inventive concepts A1-A15.
D1. A method for delivering an embolic protection device comprising:
   a corewire having a proximal section with a first diameter, a distal section having a second diameter less than the first diameter, and a tip section having a non-circular cross section over at least a portion of its length;
   a hypotube with a proximal section free of laser cuts and a distal section having laser cuts through the wall of the hypotube, wherein the corewire extends through the hypotube with the proximal end and the distal end of the corewire extending from respective ends of the hypotube;
   a friction reducing structure comprising a polymer associated with the hypotube or a mechanical element having a low friction channel, located between the corewire and a portion of the hypotube distal section;
   a torque coupler restricting rotation of the corewire and the hypotube at the torque coupler while allowing at least some sliding of the corewire within the hypotube;
   a fiber bundle comprising a bundle of fibers each having a first end and a second end, a first attachment element, and a second attachment element, wherein the first attachment element secures the first end of the polymer fibers and the second attachment element secures the second end of the fibers, wherein the first attachment element is secured to the distal section of the hypotube, wherein the fiber bundle has an undeployed configuration with the fibers aligned and a deployed configuration with the fibers bent and the corewire in a distal position in the hypotube in the undeployed configuration relative to the position in the deployed configuration; and
   a distal tip secured with the second attachment element and/or to the corewire, the method comprising:
   advancing the filter cartridge through a patient's vasculature; and
   steering direction of advancement of the filter by pushing the corewire in a distal to proximal direction to curve the hypotube along a laser cut section and orienting the curve in a desired direction.
D2. The method of inventive concept D1 wherein the steering is performed to mitigate hinderance of advancement caused by a ledge effect.
D3. The method of inventive concept D2 wherein the ledge effect is encountered with the filter in a cerebral artery.
D4. The method of inventive concept D1 wherein the filter cartridge is advanced into a cerebral artery.
D5. The method of inventive concept D1 wherein the advancing of the filter cartridge is performed simultaneously with the delivery of an aspiration catheter.
D6. The method of inventive concept D5 wherein the filter cartridge and aspiration catheter are advanced from a guide catheter, wherein the aspiration catheter comprises a proximal control structure and an engagement section that engages an inner lumen of the guide catheter, and wherein the proximal control structure extends from a proximal opening of the guide catheter and provides for advancing the aspiration catheter with its distal end extending form a distal opening of the guide catheter.
D7. The method of inventive concept D5 wherein the advancing is performed with the filter cartridge positioned to avoid ledge effects experienced by the aspiration catheter.
D8. The method of inventive concept D7 wherein the embolic protection device is partially curved from advancing the corewire in a distal direction relative to the hypotube with the filter undeployed such that the filter cartridge is more effective to avoid a ledge effect.
D9. The method of inventive concept D1 wherein the embolic protection device comprises any of the features of claims 1 to 16 or inventive concepts A1-A15.
E1. A method for assembling an integrated guide structure comprising:
   assembling components of the integrated guide structure, wherein the components comprise a hypotube having laser cuts along at least a portion of the length of the hypotube that extend through the wall of the hypotube, a corewire extending through a central lumen of the hypotube, and a central element with a central hole through which at least a portion of the corewires extends through and an outer surface that fits within the central lumen of the hypotube; and
   reflowing a thermoplastic elastomer jacket over the exterior of the laser cut hypotube, wherein thermoplastic elastomer extends through holes through the hypotube to engage the exterior of the central element to resist movement of the central element without restricting at least some movement of the corewire within the lumen of the hypotube.
E2. The method of inventive concept E1 wherein the central element is an inner coil mounted over the corewire and located within the lumen of the hypotube.
E3. The method of inventive concept E2 wherein the inner coil extends within the laser cut section from about the proximal end of the laser cut hypotube in a distal direction and wherein the corewire has a proximal section with a first diameter and a distal section with a diameter less than the first diameter, the inner coil mounted over the distal section of the corewire.
E4. The method of inventive concept E1 wherein the central element is a stake with an outer surface with a non-circular cross section, wherein the stake has a lumen with a non-circular cross section, and wherein the corewire extending through the stake has a matching non-cylindrical cross section to rotationally couple the corewire the stake, and the hypotube.
E4. The method of inventive concept E1 wherein the laser cut section of the hypotube extend along from about 15 to about 47 percent of the hypotube length.
E5. The method of inventive concept E1 wherein the laser cut section of the hypotube has an extent along from about 8 inches to about 30 inches.
E6. The method of inventive concept E1 wherein a laser cut section of the hypotube comprises an intermittent spiral cut.
E7. The method of inventive concept E1 wherein the laser cut portion of the hypotube has changes to the cut portions to increase flexibility toward the distal end of the device.
E8. The method of inventive concept E1 further comprising sliding a filter cartridge over the distal end of the corewire, securing the proximal end of the filter cartridge to the hypotube, and securing the distal end of the filter cartridge to the corewire, wherein a twist of the filter cartridge is performed in conjunction with the securing steps.
E9. The method of inventive concept E8 wherein a proximal marker band associated with the filter cartridge comprises a crimp feature that engages the corewire to couple rotational motion of the proximal marker band and the corewire,
E10. The method of inventive concept E8 wherein the proximal marker band comprises a collar with a greater diameter than adjacent sections and wherein securing the proximal marker band to the hypotube comprises laser welding the hypotube to the collar.

The embodiments above are intended to be illustrative and not limiting. Additional embodiments are within the claims. In addition, although the present invention has been described with reference to particular embodiments, those skilled in the art will recognize that changes can be made in form and detail without departing from the spirit and scope of the invention. Any incorporation by reference of documents above is limited such that no subject matter is incorporated that is contrary to the explicit disclosure herein. To the extent that specific structures, compositions and/or processes are described herein with components, elements, ingredients or other partitions, it is to be understand that the disclosure herein covers the specific embodiments, embodiments comprising the specific components, elements, ingredients, other partitions or combinations thereof as well as embodiments consisting essentially of such specific components, ingredients or other partitions or combinations thereof that can include additional features that do not change the fundamental nature of the subject matter, as suggested in the discussion, unless otherwise specifically indicated. The use of the term “about” herein refers to expected uncertainties in the associated values as would be understood in the particular context by a person of ordinary skill in the art.

What is claimed is:

1. An embolic protection device with a flexible fiber-based filter element comprising:
a corewire having a proximal section with a first diameter, a distal section having a second diameter less than the first diameter, and a tip section having a non-circular cross section over at least a portion of its length;
a hypotube with a proximal section free of laser cuts and a distal section having laser cuts through the wall of the hypotube, wherein the corewire extends through the hypotube with the proximal end and the distal end of the corewire extending from respective ends of the hypotube;
a friction reducing coil between the corewire and at least a portion of the hypotube distal section;
a torque coupler restricting rotation of the corewire and the hypotube at the torque coupler while allowing at least some sliding of the corewire within the hypotube;
a fiber bundle comprising a bundle of fibers each having a first end and a second end, a first attachment element, and a second attachment element, wherein the first attachment element secures the first end of the polymer fibers and the second attachment element secures the second end of the fibers, wherein the first attachment element is secured to the distal section of the hypotube, wherein the fiber bundle has an initial undeployed configuration with the fibers aligned and a deployed configuration with the fibers bent and the corewire in a proximal position relative to the hypotube in the undeployed configuration;
a distal tip secured with the second attachment element and/or to the corewire.

2. The embolic protection device of claim 1 wherein a stake attached to the hypotube has a channel with an asymmetry of the cross section of the channel surface around the axis of the corewire and wherein the corewire comprises a segment with an asymmetric key structure that interfaces with the channel of the stake to form the torque coupler restricting the relative rotation of the corewire and the hypotube without restricting sliding of the corewire within the hypotube over the segment of the corewire.

3. The embolic protection device of claim 2 wherein the stake is secured to the laser cut hypotube by material extending through the laser cut openings and is positioned within the laser cut hypotube proximal to the fiber cartridge.

4. The embolic protection device of claim 2 wherein the stake is also a marker band for the fiber cartridge, wherein the stake/marker band has a collar for attachment to the laser cut hypotube, a crimp section at which a lumen of the marker band has a non-cylindrical cross section forming a component of the torque coupler, a fiber attachment section, and a central section under but not attached to the fibers oriented toward the center of the fiber cartridge, wherein the crimp section is between the collar and the fiber attachment section.

5. The embolic protection device of claim 2 wherein the orientation of the first attachment element and the second attachment element provides a twist to the fiber bundle which is maintained by the torque coupler.

6. The embolic protection device of claim 1 wherein the hypotube comprises reflowed thermoplasic elastomer over at least a portion of the laser cut hypotube and wherein the reflowed thermoplastic elastomer extends into cut openings of the hypotube.

7. The embolic protection device of claim 6 wherein the reflowed thermoplastic elastomer extends through the cut openings and secures a stake forming a component of the torque coupler.

8. The embolic protection device of claim 7 wherein the fiber cartridge comprises a laser cut marker band integrated with one of the attachment structures.

9. The embolic protection device of claim 1 wherein 90% of the distal 5 inches of the hypotube is laser cut.

10. The embolic protection device of claim 1 wherein the corewire comprises an intermediate section between the proximal section, the distal section having a third diameter intermediate between the first diameter and the second diameter, wherein transition sections smoothly connect the sections, and wherein the hypotube comprises an intermediate section between the proximal section and distal section.

11. The embolic protection device of claim 1 wherein the fiber bundle comprises at least about 100 surface capillary fibers distributed around the circumference of the corewire with a twist of the distal end relative to the proximal end of at least about 90 degrees and wherein the distal tip comprises a distal coil and a blunt tip.

12. The embolic protection device of claim 1 wherein the fiber bundle comprises radiopaque metal elements.

13. The embolic protection device of claim 1 wherein the laser cut hypotube has a spiral intermittent cut.

14. The embolic protection device of claim 11 wherein the fraction of metal cut at a section near the distal end is greater than a section at a proximal end of the laser cut hypotube to introduce greater flexibility at the distal end.

15. The embolic protection device of claim 11 wherein the distal end has been mechanically stretched to increase flexibility.

16. The embolic protection device of claim 1 wherein pushing the corewire in a distal direction from an undeployed filter configuration results in bending of the device along the length of the laser cut hypotube.
